SEQUENCE LISTING (53 sequences)

SEQ ID NO: 1 - DNA, 21 bp, synthetic construct
  misc_feature 1..21: note = SOX2 5'Primer
  source 1..21: mol_type = other DNA
gggggaatggaccttgtatag

SEQ ID NO: 2 - DNA, 20 bp, synthetic construct
  misc_feature 1..20: note = SOX2 3'Primer
  source 1..20: mol_type = other DNA
gcaaagctcctaccgtacca

SEQ ID NO: 3 - DNA, 19 bp, synthetic construct
  misc_feature 1..19: note = CDX2 5'Primer
  source 1..19: mol_type = other DNA
atcaccatccggaggaaag

SEQ ID NO: 4 - DNA, 20 bp, synthetic construct
  misc_feature 1..20: note = CDX2 3'Primer
  source 1..20: mol_type = other DNA
tgcggttctgaaaccagatt

SEQ ID NO: 5 - DNA, 18 bp, synthetic construct
  misc_feature 1..18: note = PDX1 5'Primer
  source 1..18: mol_type = other DNA
aagctcacgcgtggaaag

SEQ ID NO: 6 - DNA, 22 bp, synthetic construct
  misc_feature 1..22: note = PDX1 3'Primer
  source 1..22: mol_type = other DNA
gccgtgagatgtacttgttgaa

SEQ ID NO: 7 - DNA, 19 bp, synthetic construct
  misc_feature 1..19: note = HHEX 5'Primer
  source 1..19: mol_type = other DNA
cggacggtgaacgactaca

SEQ ID NO: 8 - DNA, 21 bp, synthetic construct
  misc_feature 1..21: note = HHEX 3'Primer
  source 1..21: mol_type = other DNA
agaaggggctccagagtagag

SEQ ID NO: 9 - DNA, 20 bp, synthetic construct
  misc_feature 1..20: note = ALDH1A2 5'Primer
  source 1..20: mol_type = other DNA
ccacagtgttttccaacgtc

SEQ ID NO: 10 - DNA, 18 bp, synthetic construct
  misc_feature 1..18: note = ALDH1A2 3'Primer
  source 1..18: mol_type = other DNA
tcctgaacagggccaaag

SEQ ID NO: 11 - DNA, 18 bp, synthetic construct
  misc_feature 1..18: note = CYP26A1 5'Primer
  source 1..18: mol_type = other DNA
cgagcactcgtgggagag

SEQ ID NO: 12 - DNA, 20 bp, synthetic construct
  misc_feature 1..20: note = CYP26A1 3'Primer
  source 1..20: mol_type = other DNA
ccaaagaggagttcggttga

SEQ ID NO: 13 - DNA, 18 bp, synthetic construct
  misc_feature 1..18: note = RARA 5'Primer
  source 1..18: mol_type = other DNA
gccatctgcctcatctgc

SEQ ID NO: 14 - DNA, 20 bp, synthetic construct
  misc_feature 1..20: note = RARA 3'Primer
  source 1..20: mol_type = other DNA
tccgcacgtagacctttagc

SEQ ID NO: 15 - DNA, 18 bp, synthetic construct
  misc_feature 1..18: note = RARB 5'Primer
  source 1..18: mol_type = other DNA
ccgaaaagctcaccagga

SEQ ID NO: 16 - DNA, 19 bp, synthetic construct
  misc_feature 1..19: note = RARB 3'Primer
  source 1..19: mol_type = other DNA
cgatggtcagcactggaat

SEQ ID NO: 17 - DNA, 20 bp, synthetic construct
  misc_feature 1..20: note = RARG 5'Primer
  source 1..20: mol_type = other DNA
cagccctacatgttcccaag

SEQ ID NO: 18 - DNA, 20 bp, synthetic construct
  misc_feature 1..20: note = RARG 3'Primer
  source 1..20: mol_type = other DNA
ggcctggaatctccatcttc

SEQ ID NO: 19 - DNA, 22 bp, synthetic construct
  misc_feature 1..22: note = HES1 5'Primer
  source 1..22: mol_type = other DNA
tgccagctgatataatggagaa

SEQ ID NO: 20 - DNA, 25 bp, synthetic construct
  misc_feature 1..25: note = HES1 3'Primer
  source 1..25: mol_type = other DNA
ctccataataggctttgatgacttt

SEQ ID NO: 21 - DNA, 22 bp, synthetic construct
  misc_feature 1..22: note = INS 5'Primer
  source 1..22: mol_type = other DNA
aggcttcttctacacacccaag

SEQ ID NO: 22 - DNA, 18 bp, synthetic construct
  misc_feature 1..18: note = INS 3'Primer
  source 1..18: mol_type = other DNA
cacaatgccacgcttctg

SEQ ID NO: 23 - DNA, 19 bp, synthetic construct
  misc_feature 1..19: note = GCG 5'Primer
  source 1..19: mol_type = other DNA
gtacaaggcagctggcaac

SEQ ID NO: 24 - DNA, 21 bp, synthetic construct
  misc_feature 1..21: note = GCG 3'Primer
  source 1..21: mol_type = other DNA
tgggaagctgagaatgatctg

SEQ ID NO: 25 - DNA, 19 bp, synthetic construct
  misc_feature 1..19: note = NKX2-2 5'Primer
  source 1..19: mol_type = other DNA
cgagggccttcagtactcc

SEQ ID NO: 26 - DNA, 19 bp, synthetic construct
  misc_feature 1..19: note = NKX2-2 3'Primer
  source 1..19: mol_type = other DNA
ggggacttggagcttgagt

SEQ ID NO: 27 - DNA, 22 bp, synthetic construct
  misc_feature 1..22: note = CPA1 5'Primer
  source 1..22: mol_type = other DNA
cagcatcatcaaggcaatttat

SEQ ID NO: 28 - DNA, 19 bp, synthetic construct
  misc_feature 1..19: note = CPA1 3'Primer
  source 1..19: mol_type = other DNA
ggagctcgaaggtgaagga

SEQ ID NO: 29 - DNA, 19 bp, synthetic construct
  misc_feature 1..19: note = CEL 5'Primer
  source 1..19: mol_type = other DNA
ggagctcgaaggtgaagga

SEQ ID NO: 30 - DNA, 21 bp, synthetic construct
  misc_feature 1..21: note = CEL 3'Primer
  source 1..21: mol_type = other DNA
gatcccgaaggccatagttac

SEQ ID NO: 31 - DNA, 26 bp, synthetic construct
  misc_feature 1..26: note = CFTR 5'Primer
  source 1..26: mol_type = other DNA
gaagtagtgatggagaatgtaacagc

SEQ ID NO: 32 - DNA, 23 bp, synthetic construct
  misc_feature 1..23: note = CFTR 3'Primer
  source 1..23: mol_type = other DNA
gctttctcaaataattccccaaa

SEQ ID NO: 33 - DNA, 21 bp, synthetic construct
  misc_feature 1..21: note = CAPN6 5'Primer
  source 1..21: mol_type = other DNA
aggctgctataacaaccgtga

SEQ ID NO: 34 - DNA, 20 bp, synthetic construct
  misc_feature 1..20: note = CAPN6 3'Primer
  source 1..20: mol_type = other DNA
catcctcaggcacagtgaag

SEQ ID NO: 35 - DNA, 20 bp, synthetic construct
  misc_feature 1..20: note = ALB 5'Primer
  source 1..20: mol_type = other DNA
gtgaggttgctcatcggttt

SEQ ID NO: 36 - DNA, 20 bp, synthetic construct
  misc_feature 1..20: note = ALB 3'Primer
  source 1..20: mol_type = other DNA
gagcaaaggcaatcaacacc

SEQ ID NO: 37 - DNA, 18 bp, synthetic construct
  misc_feature 1..18: note = CYP3A7 5'Primer
  source 1..18: mol_type = other DNA
caaacttggccgtggaaa

SEQ ID NO: 38 - DNA, 20 bp, synthetic construct
  misc_feature 1..20: note = CYP3A7 3'Primer
  source 1..20: mol_type = other DNA
agtccatgtgtacgggttcc

SEQ ID NO: 39 - DNA, 21 bp, synthetic construct
  misc_feature 1..21: note = HNF4A 5'Primer
  source 1..21: mol_type = other DNA
gagatccatggtgttcaagga

SEQ ID NO: 40 - DNA, 20 bp, synthetic construct
  misc_feature 1..20: note = HNF4A 3'Primer
  source 1..20: mol_type = other DNA
gtgccgagggacaatgtagt

SEQ ID NO: 41 - DNA, 27 bp, synthetic construct
  misc_feature 1..27: note = AFP 5'Primer
  source 1..27: mol_type = other DNA
tgtactgcagagataagtttagctgac

SEQ ID NO: 42 - DNA, 23 bp, synthetic construct
  misc_feature 1..23: note = AFP 3'Primer
  source 1..23: mol_type = other DNA
tccttgtaagtggcttcttgaac

SEQ ID NO: 43 - DNA, 20 bp, synthetic construct
  misc_feature 1..20: note = APOA2 5'Primer
  source 1..20: mol_type = other DNA
gagaaggtcaagagcccaga

SEQ ID NO: 44 - DNA, 20 bp, synthetic construct
  misc_feature 1..20: note = APOA2 3'Primer
  source 1..20: mol_type = other DNA
ccttcttgatcaggggtgtc

SEQ ID NO: 45 - DNA, 43 bp, Homo sapiens
  source 1..43: mol_type = unassigned DNA
gctaccccagccagtgtcaacacgacaccggataaaccaaaga

SEQ ID NO: 46 - DNA, 25 bp, synthetic construct
  misc_feature 1..25: note = hHES1 gRNA1 F
  source 1..25: mol_type = other DNA
caccgatccggtgtcgtgttgacac

SEQ ID NO: 47 - DNA, 25 bp, synthetic construct
  misc_feature 1..25: note = hHES1 gRNA1 R
  source 1..25: mol_type = other DNA
ctaggccacagcacaactgtgcaaa

SEQ ID NO: 48 - DNA, 22 bp, synthetic construct
  misc_feature 1..22: note = hHES1 Seq F
  source 1..22: mol_type = other DNA
accctcagcacttgctcagtag

SEQ ID NO: 49 - DNA, 25 bp, synthetic construct
  misc_feature 1..25: note = hHES1 Seq R
  source 1..25: mol_type = other DNA
tcttcgtcttttctccataataggc

SEQ ID NO: 50 - DNA, 50 bp, synthetic construct
  misc_feature 1..50: note = Modified gene sequence of WT and HES1KO (Del #11)-A
  source 1..50: mol_type = other DNA
aaaaaaaaaaggaaaatgccagctgatataatggagaaaaattcctcgtc

SEQ ID NO: 51 - skipped

SEQ ID NO: 52 - DNA, 41 bp, synthetic construct
  misc_feature 1..41: note = Modified gene sequence of WT and HES1KO (Del #11)-C
  source 1..41: mol_type = other DNA
cccggtgctaccccagccagtgtcaacacgacaccggataa

SEQ ID NO: 53 - DNA, 15 bp, synthetic construct
  misc_feature 1..15: note = Modified gene sequence of WT and HES1KO (Del #11)-D
  source 1..15: mol_type = other DNA
cacgacaccggataa

(19) United States
(12) Patent Application Publication
(10) Pub. No.: US 2026/0022346 A1
(43) Pub. Date: Jan. 22, 2026

(54) HEPATO-BILIARY-PANCREATIC TISSUES AND METHODS OF MAKING SAME

(71) Applicant: Children's Hospital Medical Center, Cincinnati, OH (US)

(72) Inventors: Takanori TAKEBE, Cincinnati, OH (US); Hiroyuki KOIKE, Tokyo (JP)

(73) Assignee: Children's Hospital Medical Center, Cincinnati, OH (US)

(21) Appl. No.: 19/298,396

(22) Filed: Aug. 13, 2025

Related U.S. Application Data
(63) Continuation of application No. 17/263,124, now Pat. No. 12,421,500.
(60) Provisional application No. 62/703,559, filed on Jul. 26, 2018.

(51) Int. Cl. C12N 5/071; A61K 35/28; A61K 35/39; A61K 35/407; A61K 35/413; A61K 35/44; A61K 35/545

(52) U.S. Cl. CPC C12N 5/0677; A61K 35/28; A61K 35/39; A61K 35/407; A61K 35/413; A61K 35/44; A61K 35/545; C12N 5/0671; C12N 5/0697; C12N 2501/115; C12N 2501/155; C12N 2501/415; C12N 2501/727; C12N 2502/14; C12N 2502/22; C12N 2506/45; C12N 2513/00; C12N 2533/90

(57) ABSTRACT

Disclosed herein are hepato-biliary-pancreatic organoid (“HBPO” or “HBP organoid”) compositions, and methods of making and using hepato-biliary-pancreatic organoid compositions. The disclosed compositions may have two or more functions selected from hepatic tissue function, biliary tissue function, exocrine pancreatic function, and endocrine pancreatic tissue function. Methods of treating individuals using the hepato-biliary-pancreatic organoid compositions is also disclosed.

### CROSS-REFERENCE TO RELATED APPLICATIONS

This application is a Continuation application from U.S. application Ser. No. 17/263,124 which is the National Phase entry of International Application No. PCT/US2019/041985, filed Jul. 16, 2019, designating the United States and published in the English language, which claims priority to and benefit of U.S. Application No. 62/703,559, entitled “Modeling hepato-biliary-pancreatic organogenesis from the foregut-midgut boundary in humans,” filed Jul. 26, 2018, the contents of each of which are expressly incorporated herein by reference in their entirety for all purposes.

### REFERENCE TO SEQUENCE LISTING

The instant application contains a Sequence Listing which has been submitted electronically in XML file format and is hereby incorporated by reference in its entirety. Said XML copy, created on Aug. 6, 2025, is named CHMC_P0040US_C1_SL.xml and is 66,501 bytes in size and is hereby expressly incorporated by reference in its entirety.

### BACKGROUND

Organogenesis is a complex and inter-connected process, orchestrated by multiple boundary tissue interactions¹⁻⁷. However, to date, it has been unclear how individual, neighboring components coordinate to establish an integral multi-organ structure. Thus, multi-organ integration in stem cell culture has been a critical unmet challenge. Specifically, obtaining structurally and functionally integrated organoids having more than one tissue type is an unmet need in the art. More particularly, the patterning and balanced organogenesis of the hepato-biliary-pancreatic (HBP) system has not been successfully modelled in tissue culture due to technical complexities, hindering detailed mechanistic studies^{16,17}. The instant disclosure seeks to address one or more of the aforementioned needs in the art.

### BRIEF SUMMARY

Disclosed herein are hepato-biliary-pancreatic organoid (“HBPO” or “HBP organoid”) compositions, and methods of making and using hepato-biliary-pancreatic organoid compositions. The disclosed compositions may have two or more functions selected from hepatic tissue function, biliary tissue function, exocrine pancreatic function, and endocrine pancreatic tissue function. Methods of treating individuals using the hepato-biliary-pancreatic organoid compositions is also disclosed.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1A-1I Boundary organoid generates multi-endoderm domains. FIG. 1A. Schematic overview for establishing the hepato-biliary-pancreatic (HBP) organoid (“HBPO”) from iPSC. Human PSCs were differentiated into anterior or posterior gut cells. The cells were dissociated into single cells and reaggregated to form anterior/posterior gut spheroids, and anterior-posterior boundary organoids were generated in Matrigel. Matrigel embedding initiated multi-organ specification and invagination from boundary organoids. FIG. 1B and FIG. 1C. Generation of boundary organoid via fusion of SOX2+ anterior and CDX2+ posterior gut spheroids. SOX2 and CDX2 expressions were confirmed by wholemount immunostaining for SOX2, CDX2, and DAPI in White, flowcytometry for percentage of each ⁻ cells in each area compared to DAPI stained total cell numbers. FIG. 1G. Percentage of HHEX and PDX1 positive cells for fused organoid in various combination, anterior-posterior (AP)(n=4), anterior-anterior (AA)(n=3) and posterior-posterior (PP)(n=3) at Day 11. Y axis showed percentages of positive cells compared to DAPI stained total cell numbers. Data are mean±s.d. * P<0.05, ** P<0.01; one-way ANOVA. FIG. 1H and FIG. 1I Transcriptomic characterization of boundary organoids with time course, from Day 8 (D8) to Day 12 (D12). Anterior (A), boundary (B), and posterior (P) domains were dissected, separated, and applied for RNA sequencing as indicated in left representative image (anterior gut spheroid was labeled by GFP and posterior gut spheroid was labeled by RFP). Anterior, boundary, and posterior domains showed the enrichment of the gene-sets of anterior foregut, liver/biliary/pancreas primordium and mid/hindgut markers reported 31.32, respectively. Scale bars, 50 μm (FIG. 1B), 100 μm (FIG. 1C), 50 μm (FIG. 1H).

FIG. 2A-21. Self-emergence of hepato-biliary-pancreatic progenitors from boundary organoid without inductive factors. FIG. 2A. Generating PROX1-tdTomato reporter line by CRISPR-Cas9 gene editing system. FIG. 2B. PROX1-tdTomato expression in established PROX1 reporter iPSCs from Day 9 to Day 11. FIG. 2C. PROX1-tdTomato positive area in each organoid. AP, AA and PP combinations were evaluated. The tdTomato expression was confirmed in the cell membrane of boundary organoids in AP combination. FIG. 2D. Hepatic invagination in mouse liver primordium explant (Prox1::GFP) and human iPSC-derived boundary organoids (PROX1::m-tdTomato). FIG. 2E. The immunostaining of PROX1 in E8.75 mice embryo and boundary organoids at Day 13. FIG. 2F. Transcriptomic characterization of boundary organoids. Anterior, boundary, and posterior domains were dissected, separated, and applied for RNA sequencing. Heatmap shows downstream gene expression related to FGF, BMP, Hedgehog, NOTCH and RA signal pathway selected from GO term category and KEGG pathway category. Heatmap was separated into 8 detailed groups (C1-C8) by unbiased hierarchical clustering. C6 which showed unique expression pattern of highly expressed genes in B indicated an enrichment of RA down-stream gene-sets compared to others. FIG. 2G. Gene expression of HHEX, PDX1, SOX2, and CDX2 at Day 11 with three days culture of retinoic acid, BMS493, R-75251, or WIN18446. 2H. Gene expression analysis for RA signal pathway related genes in epithelial or mesenchymal cells from original anterior or posterior gut spheroids. Each gut spheroids were differentiated using RFP or GFP labeled iPSCs and dissociated into single cells after boundary formation. Anterior/Posterior separation was performed by RFP or GFP expression, whereas epithelial/mesenchymal separation was by EpCAM expression. FIG. 2I. Default developmental potential of transplanted boundary organoid. Middle panels show H&E staining and immunohistochemistry analysis, whereas right panels show immunofluorescent analysis. Scale bars, 50 μm (FIG. 2B), 200 μm (FIG. 2D), 100 μm (FIG. 2E), 100 μm (FIG. 2I).

FIG. 3A-FIG. 3R. Modeling human hepato-biliary-pancreatic organogenesis. FIG. 3A. Illustration of dissection of PROX1 positive region from HBP organoids and image of before/after dissection. FIG. 3B. Optimization of cultured system with 1) Floating, 2) Embedded into Matrigel, 3) Embedded into Matrigel and cultured with Transwell from D13, 4). Dissected, embedded into Matrigel, and cultured with Transwell from D13. Left panel shows the classification of invaginating or branching organoid. FIG. 3C. Morphogenesis of boundary organoids through 2 days from Day 13. FIG. 3D. Morphogenetical change of PROX1 dissected tissue from boundary organoids through 30 days of air-liquid interface cultured system. FIG. 3E. Stereomicroscopic image of Day 37 organoids. FIG. 3F. Boundary organoid has tdTomato expressing hepatobiliary tissues branched out for putative pancreatic domains, similar to cultured mouse E10.5 derived hepato-biliary-pancreas. Left: cultured mouse embryonic tissue during 4 days, Right: PROX1-tdTomato reporter iPSCs at Day 90. FIG. 3G. Right: Illustration of invagination liver, bile duct and pancreas connected with intestine. Left: H&E in D90 boundary tissue. FIG. 3H-FIG. 3I. Immunostaining for combination of CK19, PDX1, PROX1, SOX9 and NGN3, and alpha-SMA and SOX17 (3H) and AFP, EpCAM, and alpha-SMA (FIG. 3I). FIG. 3J, FIG. 3K and FIG. 3L. Whole mount staining of PDX1, NKX6.1 and GATA4, and DBA, PDX1, and PROX1 (FIG. 3K and FIG. 3L). FIG. 3M and FIG. 3P. Immunostaining of NKX6.1 and HNFIB (FIG. M), Amylase and GATA4 (FIG. N and O) and Amylase and CCKAR (FIG. 3P). FIG. 3Q. CCK treatment response in putative biliary structure. FIG. 3R. Hormone induced secretory function of exocrine pancreatic domain. Enzyme-linked immunosorbent assay of amylase in boundary tissue before and after 3 days-CCK. Scale bars, 100 μm (a), 100 μm (FIG. 3B), 100 μm (FIG. 3C), 200 μm (FIG. 3D), 1 mm (FIG. 3E), 200 μm (FIG. 3F), 200 μm (FIG. 3G), 200 μm (FIG. 3H), 200 μm (FIG. 3I), 200 μm (FIG. 3J), 100 μm (FIG. 3L), 200 μm (FIG. 3M), 100 μm (FIG. 3N), 500 μm (FIG. 3P), 50 μm (FIG. 3Q).

FIG. 4A-4J. Modeling HES1-mediated organ segregation error in HBP organoids. FIG. 4A. Gene targeting strategy for HES1 knock out (KO) line by CRISPR-Cas9 system. FIG. 4B. Confirmation of modified gene sequence of WT and HES1KO (Del #11) FIG. 4C. Photo of HES1−/−iPSC culture FIG. 4D. Gene expression of HES1 in HES1 KO iPSC-derived boundary organoid at Day 20. FIG. 4E. Confirmation of boundary organoid formation from HES1−/−iPS line by wholemount immunostaining of SOX2, CDX2, PDX1 and HHES. Hepato-biliary-pancreatic precursor specification was preserved even in the presence of HES1 mutation. FIG. 4F. PROX1-tdTomato expression in anterior and posterior boundary spheroids generated from HES1+/+ and HES1−/− at D11. FIG. 4G. RNAseq of pancreas associated markers at Day 22 of HES1+/+ and HES1−/− HBP organoids. FIG. 4H. Gene expression of GCG, NEUROG3, INS, and NKX2-2 in HES1+/+, HES1−/−organoids, and human adult pancreatic tissue. FIG. 4I. Macroscopic observation of boundary tissue of HES1+/+ and HES1−/−. FIG. 4J. Wholemount immunostaining for DBA and PDX1 shows enhanced PDX1 expression and diminished DBA stained area in HES1 KO organoids. Scale bars, 500 μm (FIG. 4C), 200 μm (FIG. 4E), 100 μm (FIG. 4F), 500 μm (FIG. 4I), 500 μm (FIG. 4J).

FIG. 5. Anterior and posterior gut cell characterization. Flow cytometry of EpCAM in Day 7 anterior and posterior gut cells using TkDA human iPSCs and 72_3 human iPSCs.

FIG. 6A-6B Reproducibility of boundary organoid formation. FIG. 6A. The image of D11 boundary organoids. Anterior and posterior gut spheroids were differentiated from H1 ESCs or 1383D6 iPSCs, mixed and transferred into Matrigel. Scale bar is 200 μm. FIG. 6B. Immunofluorescent staining of CDX2, epithelial marker ECAD, and HHEX in boundary spheroids derived from H1ESC. FIG. 6C. Immunofluorescent staining of PDX1, CDX2, FOXF1, and HHEX in boundary spheroids derived from 72_3 iPS.

FIG. 7A-7B Cell-cell contact dependent HBP gene induction. FIG. 7A. Anterior and posterior gut spheroids were mixed at D8, fused the following day at D9, cultured and collected at D12 for quantitative RT-PCR. The spheroids that not fused were also collected at D12 for comparison. FIG. 7B. PDX1 and HHEX gene expressions in the condition of fused, not-fused, posterior gut spheroid (day8), and iPS cells.

FIG. 8 Comparison of different anterior and posterior gut combinations. Immunofluorescent staining of CDX2, HHEX, and PDX1 in the combination of AP, AA, and PP spheroids at D12. Scale bar is 200 μm.

FIG. 9A-9C. HBP progenitors develop from posterior gut cells. FIG. 9A. Non labeled iPS cells were differentiated into anterior gut spheroid while AAVS1-GFP labeled iPS cells were differentiated into posterior gut spheroid. Top column showed bright field and GFP fluorescent image during boundary organoid formation. Bottom column showed whole mount immunostaining for HHEX and PDX1 at Day 13. The HHEX expression was overlapped with GFP expression. Scale bar is 200 μm. FIG. 9B. H2B-GFP labeled and unlabeled PROX1-tdTomato reporter iPSCs were differentiated into anterior and posterior gut spheroid, respectively. tdTomato expression was only detected in unlabeled original posterior gut spheroid. Scale bar is 200 μm. FIG. 9C. Using unlabeled iPSCs and PROX1-tdTomato reporter iPSCs, anterior and posterior gut spheroids were differentiated. Two combinations, reporter cell derived anterior and unlabeled cell posterior (left column), or unlabeled cell derived anterior and reporter cell derived posterior gut spheroid (right column) were examined by tdTomato expression. Top row: bright field image, bottom row: tdTomato fluorescence image. Scale bar is 200 μm

FIG. 10. Abolishment of HHEX and PDX1 induction in posterior gut specific BMS493. BMS493 pretreated anterior or posterior gut spheroids were fused to induce HBP anlage formation. Compared to untreated control group, the group of BMS493 pretreated posterior gut spheroid was inhibited HHEX and PDX1 expression at boundary, suggesting retinoic acid receptor function in posterior side was important to establish HBP boundary organoid. Scale bar: 200 μm

FIG. 11A and FIG. 11B. PROX1 inhibition by BMS493 exposure with E9.0 PROX1::GFP reporter mouse embryo explant culture. Embryonic Day 9.0 Prox 1-GFP whole embryo was cultured in the rotator-type bottle culture system for 24 hrs. Retinoic acid receptors antagonist BMS493 treated group was compared with control (adding DMSO) group. FIG. 11A. Bright field image and GFP fluorescent image for embryo after culture. FIG. 11B. The area of GFP expressing parts was quantified from GFP image in (a). Scale bar: 1 mm

FIG. 12 Optimization of in vitro culture system. In FIG. 3A-3C, various culture formats were compared to enhance morphological change, such as invagination and branching morphogenesis, of PROX1 positive HBP precursor region. At D7, anterior and posterior gut spheroids were mixed and connected after 24 hour-culture. Connected spheroids were transferred into Matrigel drop or low binding culture plate to compare between non-floating and floating conditions during HBP precursor emergence. The organoid in Matrigel embedded group was started to express tdTomato at D11. The tdTomato positive region was manually dissected under microscope according to the fluorescence expression and transferred into Matrigel drop again or Transwell to compare the effect from various agonist and antagonist in medium.

FIG. 13. Comparison of organoid size, PROX1 positive area, branching and invagination. Only AP combination increased the size of the organoids and PROX1 expressing region. Moreover, AP combination showed the spheroids with branching and invagination while other two combination did not. Scale bar: 500 μm

FIG. 14. Failure to branch and invaginate from posterior region of HBP organoids. While HBP organoid formed Prox 1 expressing branching structure, posterior region of HBP that contain PDX1 expression did not form its structure. Scale bar is 200 μm.

FIG. 15A-15C. Expression of organ domain-specific markers in HBP organoids. FIG. 15A. Immunofluorescent staining of AFP, Albumin, and HHEX at Day 30. AFP and Albumin expressed in the same region but not HHEX. HHEX were hepatocyte progenitor marker which result in disappearance of the expression at the later stage. FIG. 15B. Immunofluorescent staining of NKX6.1, NKX6.3 and PDX1. NKx6.3 were expressed in the area of pancreatic markers PDX1 and NKX6.1 expression. FIG. 15C. Immunofluorescent staining of EpCAM, PROX1, SOX9, and CLF. Scale bar: 100 μm

FIG. 16. Pancreatic associated genes were upregulated in HES−/− organoids. RNAseq of pancreatic associated markers at Day 22 of HES1+/+ and HES1−/− HBP organoids. This is related to FIG. 4G.

FIG. 17. Connected structure in long term cultured organoid. Whole mount staining of DBA and SOX9 in HES1−/− and HES1+/+ organoids. DBA and SOX9 disappeared in HES1−/− organoids. Scale bar: 200 μm.

### DETAILED DESCRIPTION

### Definitions

Unless otherwise noted, terms are to be understood according to conventional usage by those of ordinary skill in the relevant art. In case of conflict, the present document, including definitions, will control. Preferred methods and materials are described below, although methods and materials similar or equivalent to those described herein may be used in practice or testing of the present invention. All publications, patent applications, patents and other references mentioned herein are incorporated by reference in their entirety. The materials, methods, and examples disclosed herein are illustrative only and not intended to be limiting.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20%, or up to 10%, or up to 5%, or up to 1% of a given value. Alternatively, particularly with respect to biological systems or processes, the term may mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the term “definitive endoderm (DE) cell” means one of the three primary germ layers produced by the process of gastrulation.

As used herein the term “wnt signalling pathway” means the wnt/beta-catenin pathway and is a signal transduction pathway that is mediated by Wnt ligands and frizzled cell surface receptors that acts through the beta-catenin protein.

As used herein the term “activator” with respect to a pathway, such as a “wnt pathway” means a substance that activates the Wnt/beta-catenin pathway such that Wnt/beta-catenin targets are increased.

As used herein, the term “FGF signaling pathway activator” means a substance that activates the FGF pathway such that FGF targets are increased.

As used herein, the term “BMP signaling pathway inhibitor” a substance that interferes with the BMP pathway and causes BMP targets to be decreased.

As used herein, the term “growth factor” means a substance capable of stimulating cellular processes including but not limited to growth, proliferation, morphogenesis or differentiation.

As used herein, the term “stable expression” of a marker means expression that does not change upon modification of the growth environment.

As used herein, the term “totipotent stem cells” (also known as omnipotent stem cells) are stem cells that can differentiate into embryonic and extra-embryonic cell types. Such cells can construct a complete, viable, organism. These cells are produced from the fusion of an egg and sperm cell. Cells produced by the first few divisions of the fertilized egg are also totipotent.

As used herein, the term “pluripotent stem cells (PSCs),” also commonly known as PS cells, encompasses any cells that can differentiate into nearly all cells, i.e., cells derived from any of the three germ layers (germinal epithelium), including endoderm (interior stomach lining, gastrointestinal tract, the lungs), mesoderm (muscle, bone, blood, urogenital), and ectoderm (epidermal tissues and nervous system). PSCs can be the descendants of totipotent cells, derived from embryos (including embryonic germ cells) or obtained through induction of a non-pluripotent cell, such as an adult somatic cell, by forcing the expression of certain genes.

As used herein, the term “induced pluripotent stem cells (iPSCs),” also commonly abbreviated as iPS cells, refers to a type of pluripotent stem cells artificially derived from a normally non-pluripotent cell, such as an adult somatic cell, by inducing a “forced” expression of certain genes.

As used herein, the term “precursor cell” encompasses any cells that can be used in methods described herein, through which one or more precursor cells acquire the ability to renew itself or differentiate into one or more specialized cell types. In some aspects, a precursor cell is pluripotent or has the capacity to becoming pluripotent. In some aspects, the precursor cells are subjected to the treatment of external factors (e.g., growth factors) to acquire pluripotency. In some aspects, a precursor cell can be a totipotent stem cell; a pluripotent stem cell (induced or non-induced); a multipotent stem cell; and a unipotent stem cell. In some aspects, a precursor cell can be from an embryo, an infant, a child, or an adult. In some aspects, a precursor cell can be a somatic cell subject to treatment such that pluripotency is conferred via genetic manipulation or protein/peptide treatment.

In developmental biology, cellular differentiation is the process by which a less specialized cell becomes a more specialized cell type. As used herein, the term “directed differentiation” describes a process through which a less specialized cell becomes a particular specialized target cell type. The particularity of the specialized target cell type can be determined by any applicable methods that can be used to define or alter the destiny of the initial cell. Exemplary methods include but are not limited to genetic manipulation, chemical treatment, protein treatment, and nucleic acid treatment.

Disclosed herein are hepato-biliary-pancreatic organoid (“HBPO” or “HBP organoid”) compositions. The disclosed HBPO compositions may have, in some aspects, two or more functions selected from hepatic tissue function, biliary tissue function, exocrine pancreatic function, and endocrine pancreatic tissue function. In one aspect, the HBPO disclosed herein may be referred to interchangeably as a “multi-organ three-dimensional organoid” and may comprise an anterior region, a posterior region and a boundary region. In one aspect, the boundary region may expresse Pancreatic and Duodenal Homeobox 1 (PDX1) and Hematopoietically-Expressed Homeobox Protein (HHEX). In one aspect, the HBPO may comprise bile duct tissue and pancreatic tissue. In one aspect, the bile duct tissue and pancreatic tissue may be connected in the HBPO. In one aspect, the HBPO may comprise liver cells, pancreas cells, bile duct cells, and intestinal cells.

In one aspect, the HBPO may comprise endothelial cells, mesenchymal cells, or both endothelial cells and mesenchymal cells. The HBPO may comprise, in certain aspects, endoderm and mesoderm.

The disclosed HBPOs may be characterized by a branched structure, as shown, for example, in the corresponding figures herein.

In one aspect, the HBPOs disclosed herein may be characterized by expression of a functional exocrine marker. In one aspect, the marker may be amylase. In one aspect, the marker may be an endocrine marker, for example, insulin.

In certain aspects, the HBPOs disclosed herein may be characterized by a functional response to an exogenously administered agent. For example, in one aspect, the HBPO may secrete amylase in response to contact with cholecystokinin (CCK).

In certain aspects, the disclosed HBPOs may be characterized in that the HBPO may be substantially free of one or more of submucosal glands, transition zones, vasculature, immune cells, or submucosal layers. Such features distinguish the three-dimensional organoid structure from that of native tissue found endogenously in a human, or other mammal.

In one aspect, the HBPO is obtained by expansion of one or more precursor cells as defined above, for example, an iPSC obtained from an individual.

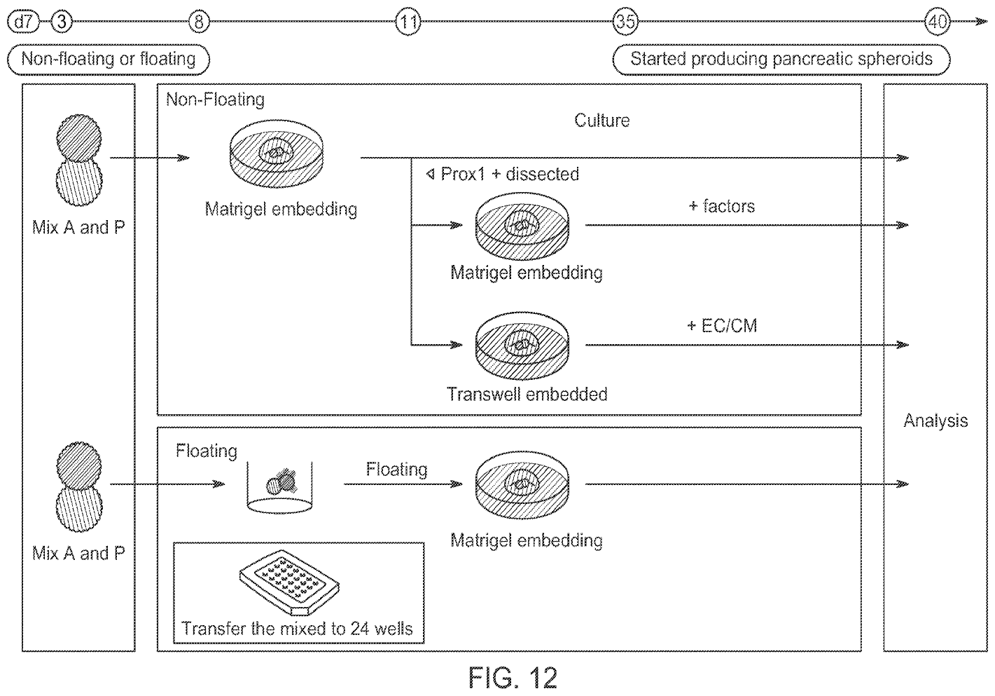

Also disclosed herein are methods of making a hepato-biliary-pancreatic organoid (“HBPO” or “HBP organoid”). In this aspect, the method may comprise contacting a first definitive endoderm with a Wnt signaling pathway activator, an FGF signaling pathway activator, and a BMP signaling pathway inhibitor to form an anterior gut spheroid; contacting a second definitive endoderm with a Wnt signaling pathway activator and an FGF signaling pathway activator to form a posterior gut spheroid; contacting the anterior gut spheroid with the posterior gut spheroid until the anterior gut spheroid and the posterior gut spheroid are fused to form a boundary organoid having a foregut-midgut boundary; and culturing the boundary organoid having the foregut-midgut boundary to form the HBPO; wherein the HBPO comprises biliary tissue and pancreatic tissue. (See, for example, FIG. 12) In one aspect, the disclosed methods may allow for the production of an HBPO that may comprise liver tissue, pancreatic tissue, bile duct tissue, and intestinal tissue.

As used herein, the term hepato-biliary-pancreatic organoid (“HBPO” or “HBP organoid”) generally refers to the organoid at the stage in which multi-organ domains (hepatic, biliary and pancreatic organ domains) are segregated, which usually occurs around D30. With regard to the term “boundary organoid,” this may include an organoid after D7, which includes organoid compositions that do not yet have tissue specification.

In one aspect, the anterior gut spheroids may comprise cells expressing SRY-box 2 (SOX2). In one aspect, the anterior gut spheroid may be characterized by SRY-box 2 (SOX2) expression. In one aspect, the anterior gut spheroids may be substantially free of Pancreatic and Duodenal Homeobox 1 (PDX1) expressing cells. In one aspect, the posterior gut spheroids comprise cells expressing Pancreatic and Duodenal Homeobox 1 (PDX1). In one aspect, the posterior gut spheroids may express CDX2. In one aspect, the posterior gut spheroid may be characterized by Caudal Type Homeobox 2 (CDX2) expression. In one aspect, the posterior gut spheroids may comprise cells expressing Caudal type homeobox 2 (CDX2). In one aspect, the HBPO may comprise cells expressing Pancreatic and Duodenal Homeobox 1 (PDX1), cells expressing Hematopoietically-expressed homeobox protein (HHEX), and cells expressing Prospero-Related Homeobox 1 (PROX1). In one aspect, the fused anterior gut spheroid and posterior gut spheroid may comprise a biliary-pancreatic primordium characterized by expression of SOX2, CDX2, HHEX, and PDX1, wherein PDX1 expression is localized to the boundary region of the fused anterior gut spheroid and the posterior gut spheroid.

In one aspect, the HBPO may be embedded into a morphogenic factor, preferably a basement membrane matrix, such as, for example, Matrigel. The method may further comprise excising PROX1 positive regions from the HBPO and culturing the excised HBPO to form invaginating epithelium and a branching structure.

In one aspect, the HBPO may be cultured with rotation, for example, using the methods disclosed herein.

In one aspect, the disclosed methods may be used to obtain an HBPO as described herein, in particular, an HBPO comprising endoderm and mesoderm.

In one aspect, the definitive endoderm may be derived from a precursor cell selected from an embryonic stem cell, an embryonic germ cell, an induced pluripotent stem cell, a mesoderm cell, a definitive endoderm cell, a posterior endoderm cell, a posterior endoderm cell, and a hindgut cell, preferably a definitive endoderm derived from a pluripotent stem cell, more preferably a definitive endoderm derived from a pluripotent stem cell selected from an embryonic stem cell, an adult stem cell, or an induced pluripotent stem cell.

In one aspect, the definitive endoderm may be derived from contacting a pluripotent stem cell with one or more molecules selected from Activin, the BMP subgroups of the TGF-beta superfamily of growth factors; Nodal, Activin A, Activin B, BMP4, Wnt3a, and combinations thereof.

In one aspect, the WNT signaling pathway activator may be selected from one or more molecules selected from the group consisting of Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, Wnt16, a GSKB inhibitor (e.g., CHIR99021, i.e. “CHIRON”), BIO, LY2090314, SB-216763, lithium, porcupine inhibitors IWP, LGK974, C59, SFRP inhibitor WAY-316606, beta-catenin activator DCA.

In one aspect, the FGF signaling pathway activator may be selected from one or more molecules selected from the group consisting of FGF1, FGF2, FGF3, FGF4, FGF10, FGF11, FGF12, FGF13, FGF14, FGF15, FGF16, FGF17, FGF18, FGF19, FGF20, FGF21, FGF22, FGF23, and combinations thereof, preferably FGF4 or FGF10, or a combination thereof.

In one aspect, the BMP signaling pathway inhibitor may be selected from Noggin, Dorsomorphin, LDN189, DMH-1, and combinations thereof, preferably wherein the BMP signaling pathway inhibitor is Noggin.

In one aspect, the disclosed methods may be conducted in vitro. In certain aspect, at least one step of the method may be carried out on a solid support. For example, the solid support may be selected from collagen, basement membrane matrix (Matrigel), or a combination thereof.

In one aspect, a method for making an HBPO having an endothelium is disclosed. In this aspect, the method may comprise the step of culturing an HBPO according to the methods disclosed herein, with endothelial cells derived from induced pluripotent stem cells. The endothelial cells may be formed into an endothelial cell (EC) spheroid prior to culturing with an HBPO. For example, for endothelial cell (EC) differentiation, human iPSCs may be dissociated using Accutase (Thermo Fisher Scientific Inc., Waltham, MA, USA) and plated on Laminin 511 E8 fragment (iMatrix-511™, provided by Nippi, Inc.) at varying optimal density (depending on the cell line) in StemFit® (Ajinomoto Co., Inc.) with Rho-associated kinase inhibitor (Y-27632). From the next day, they may first be differentiated into mesoderm using the Priming Medium having B27 medium (1:1 mixture of DMEM: F12 (1:1) with 1% Glutamax and 1% B27 (all Life Technologies) with Wnt activator and bone morphogenetic protein 4 (BMP4) for three days. The priming medium may then be replaced with EC induction medium, StemPro-34 SFM medium (Life Technologies) supplemented with VEGF and forskolin. The induction medium may be renewed daily. On day seven of differentiation, ECs are dissociated and subjected to FACS analysis. iPSC derived ECs should display typical endothelial morphology with junctional localization of CD144 and CD31. ECs may be re-plated on Fibronectin coated dishes at a density of 50,000 cells cm-2 in EC Expansion Medium consisting of StemPro-34 SFM supplemented with VEGF-A. EC Expansion Medium may be replaced every other day. The differentiated EC may be dissociated to single cells and formed into spheroids using the spheroid formation protocol of the anterior gut spheroids and posterior gut spheroids as described herein. The ECs may then be mixed with the fused anterior gut spheroid and the posterior gut spheroid on 96 well round bottom ultra-low attachment plate in gut growth medium for 24 hours to form fused EC spheroid, the anterior gut spheroid and the posterior gut spheroid.

In one aspect, a method for making an HBPO having a mesenchyme is disclosed. In this aspect, the method may comprise the step of culturing an HBPO according to a method as disclosed herein with mesenchymal cells derived from induced pluripotent stem cells. The mesenchyme cells may be formed into a mesenchyme cell (MC) spheroid prior to culturing with the HBPO. The disclosed multi-organ, three-dimensional organoid and mesenchymal cells or a mesenchymal spheroid may be cultured together to improve the growth and maturation of the multi-organ, three-dimensional organoid. For example, for mesenchyme (MSC) differentiation, human iPSCs may be differentiated to mesoderm. The mesoderm cells may then be exposed to 3 days of additional Activin A and PDGFBB. Differentiated MSC may then be dissociated to single cells and formed into spheroids using the spheroid formation protocol of the anterior gut spheroid and posterior gut spheroid. The MSC spheroid may then be mixed with the fused anterior gut spheroid and posterior gut spheroid on 96 well round bottom ultra-low attachment plate in gut growth medium for 24 hours to form a fused MSC spheroid, anterior gut spheroid and posterior gut spheroid.

In one aspect, a method of making a hepato-biliary-pancreatic organoid (“HBPO”) is disclosed, wherein the method may comprise deriving an anterior gut spheroid from anterior gut cells and a deriving a posterior gut spheroid from posterior gut cells to make a boundary organoid; and culturing the boundary organoid in gut growth medium to make the HBPO. In one aspect, a method of making a hepato-biliary-pancreatic organoid (“HBPO”), may comprise deriving an anterior gut spheroid and deriving a posterior gut spheroid from anterior gut cells and posterior gut cells to make the HBPO in the absence of inducing factors; and culturing the boundary organoids in a gut growth medium to make HBPO.

By “inducing factors,” it is meant a factor used to direct differentiation, for example, retinoic acid (RA; Sigma, MO, USA), hepatocyte growth factor (HGF; PeproTech, NJ, USA), 0.1 μM Dexamethasone (Dex; Sigma) and 20 ng/mL Oncostatin M (OSM; R&D Systems) for hepatocyte differentiation. Many other inducing factors are known for pancreas and biliary also, and would be readily understood by one of skill in the art.

In one aspect, a of method of making a hepato-biliary-pancreatic organoid (“HBPO”) is disclosed, wherein the method may comprise culturing a definitive endoderm (DE) differentiated from a pluripotent stem cell (PSC) in gut growth medium to make anterior gut cells and posterior gut cells, making an anterior gut spheroid from anterior gut cells and a posterior gut spheroid from posterior gut cells to make a boundary organoid in the absence of inducing factors; and (iii) culturing boundary organoids in gut growth medium to make an HBPO. In one aspect, the gut growth medium may be a medium comprising a ROCK inhibitor. In one aspect, the ROCK inhibitor may be Y-27632. In one aspect, the growth medium is Advanced DMEM/F-12 (Dulbecco's Modified Eagle Medium/Ham's F-12), a widely used basal medium that allows the culture of mammalian cells with reduced Fetal Bovine Serum (FBS) supplementation, available at https://www.thermofisher.com/order/catalog/product/12634010, with addition of Glutamine, HEPES, N2 and B27. In one aspect, the media may include addition of the following cytokines to Advanced DMEM: Noggin (BMP inhibitor), CHIR (WNT activator), FGF4 for anterior gut cells, CHIR and FGF4 (without Noggin) for posterior gut cells.

In one aspect, a machine for producing an HBPO is disclosed herein. The machine may comprise a gut growth medium containing a Wnt signaling pathway activator and an FGF signaling pathway activator, in the presence or absence of a BMP signaling pathway inhibitor; and a gut growth medium containing a ROCK inhibitor.

In one aspect, use of a plurality of culture mediums for the production of an HBPO, is disclosed, wherein the plurality of culture mediums comprise a gut growth medium containing a Wnt signaling pathway activator and an FGF signaling pathway activator, optionally comprising a BMP signaling pathway inhibitor; and an intestinal growth medium containing a ROCK inhibitor.

In one aspect, the HBPO may be transplanted into a mammal, such as a non-human mammal, for a period of time to increase growth and maturation of the HBPO. In one aspect, the HBPO may be transplanted to rescue organ dysfunction, for example, in an individual in need thereof. In one example, In vitro generated HBPOs may be collected and transplanted into the subscapular site of kidney or mesentery of the small intestine in non-obese diabetic/severe combined immunodeficient (NOD/SCID) mice. An increase in growth of the organoid may be observed at 8 weeks after the transplant. The viability and growth can be monitored based on blood tests such as human-Alb, human-C-peptide levels.

In one aspect, a method of treating an individual, comprising transplanting an HBPO as described herein is disclosed. In this aspect, an organoid produced according to any of the methods used herein, may be transplanted into an individual in need thereof, using standard surgical procedures as known in the art. In one aspect, the individual may have a disease state selected from hepatic disease, such as liver failure and congenital liver diseases, pancreatic disease, such as diabetes, or biliary disease.

In one aspect, methods of identifying a treatment for one or more disease states selected from a biliary inflammatory disease and/or a pancreatic inflammatory disease such as pancreatitis is disclosed. In this aspect, the disclosed organoid compositions may be contacted with a potential therapeutic agent of interest with an organoid composition as disclosed herein. A measure of organ activity may then be detected in the organoid. Based on the output of this detection, it may be determined whether the potential therapeutic agent of interest improves the measure of the disease state, and may thereby be used to identify therapeutic agents likely to be useful in treating a disease state which involves a malfunction of any of the tissues embodied in the HBPO.

Pluripotent Stem Cells Derived from Embryonic Cells

In some aspects, one step may include obtaining stem cells that are pluripotent or that can be induced to become pluripotent. In some aspects, pluripotent stem cells are derived from embryonic stem cells, which are in turn derived from totipotent cells of the early mammalian embryo and are capable of unlimited, undifferentiated proliferation in vitro. Embryonic stem cells are pluripotent stem cells derived from the inner cell mass of the blastocyst, an early-stage embryo. Methods for deriving embryonic stem cells from blastocytes are well known in the art. Human embryonic stem cells H9 (H9-hESCs) are used in the exemplary embodiments described in the present application, but it would be understood by one of skill in the art that the methods and systems described herein are applicable to any stem cells.

Additional stem cells that can be used in embodiments in accordance with the present invention include but are not limited to those provided by or described in the database hosted by the National Stem Cell Bank (NSCB), Human Embryonic Stem Cell Research Center at the University of California, San Francisco (UCSF); WISC cell Bank at the Wi Cell Research Institute; the University of Wisconsin Stem Cell and Regenerative Medicine Center (UW-SCRMC); Novocell, Inc. (San Diego, Calif.); Cellartis AB (Goteborg, Sweden); ES Cell International Pte Ltd (Singapore); Technion at the Israel Institute of Technology (Haifa, Israel); and the Stem Cell Database hosted by Princeton University and the University of Pennsylvania. Exemplary embryonic stem cells that can be used in embodiments in accordance with the present invention include but are not limited to SA01 (SA001); SA02 (SA002); ES01 (HES-1); ES02 (HES-2); ES03 (HES-3); ES04 (HES-4); ES05 (HES-5); ES06 (HES-6); BG01 (BGN-01); BG02 (BGN-02); BG03 (BGN-03); TE03 (13); TE04 (14); TE06 (16); UC01 (HSF1); UC06 (HSF6); WA01 (H1); WA07 (H7); WA09 (H9); WA13 (H13); WA14 (H14). More details on embryonic stem cells can be found in, for example, Thomson et al., 1998, “Embryonic Stem Cell Lines Derived from Human Blastocysts,” Science 282 (5391): 1145-1147; Andrews et al., 2005, “Embryonic stem (ES) cells and embryonal carcinoma (EC) cells: opposite sides of the same coin,” Biochem Soc Trans 33:1526-1530; Martin 1980, “Teratocarcinomas and mammalian embryogenesis,”. Science 209 (4458): 768-776; Evans and Kaufman, 1981, “Establishment in culture of pluripotent cells from mouse embryos,” Nature 292 (5819): 154-156; Klimanskaya et al., 2005, “Human embryonic stem cells derived without feeder cells,” Lancet 365 (9471): 1636-1641; each of which is hereby incorporated herein in its entirety.

Induced Pluripotent Stem Cells (iPSCs)

In some aspects, iPSCs are derived by transfection of certain stem cell-associated genes into non-pluripotent cells, such as adult fibroblasts. Transfection may be achieved through viral vectors, such as retroviruses. Transfected genes include the master transcriptional regulators Oct-3/4 (Pouf51) and Sox2, although it is suggested that other genes enhance the efficiency of induction. After 3-4 weeks, small numbers of transfected cells begin to become morphologically and biochemically similar to pluripotent stem cells, and are typically isolated through morphological selection, doubling time, or through a reporter gene and antibiotic selection. As used herein, iPSCs include but are not limited to first generation iPSCs, second generation iPSCs in mice, and human induced pluripotent stem cells. In some aspects, a retroviral system is used to transform human fibroblasts into pluripotent stem cells using four pivotal genes: Oct3/4, Sox2, Klf4, and c-Myc. In alternative aspects, a lentiviral system is used to transform somatic cells with OCT4, SOX2, NANOG, and LIN28. Genes whose expression are induced in iPSCs include but are not limited to Oct-3/4 (e.g., Pou5f1); certain members of the Sox gene family (e.g., Sox1, Sox2, Sox3, and Sox 15); certain members of the Klf family (e.g., Klf1, Klf2, Klf4, and Klf5), certain members of the Myc family (e.g., C-myc, L-myc, and N-myc), Nanog, and LIN28.

In some aspects, non-viral based technologies are employed to generate iPSCs. In some aspects, an adenovirus can be used to transport the requisite four genes into the DNA of skin and liver cells of mice, resulting in cells identical to embryonic stem cells. Since the adenovirus does not combine any of its own genes with the targeted host, the danger of creating tumors is eliminated. In some aspects, reprogramming can be accomplished via plasmid without any virus transfection system at all, although at very low efficiencies. In other aspects, direct delivery of proteins is used to generate iPSCs, thus eliminating the need for viruses or genetic modification. In some embodiment, generation of mouse iPSCs is possible using a similar methodology: a repeated treatment of the cells with certain proteins channeled into the cells via poly-arginine anchors was sufficient to induce pluripotency. In some aspects, the expression of pluripotency induction genes can also be increased by treating somatic cells with FGF2 under low oxygen conditions.

More details on embryonic stem cells can be found in, for example, Kaji et al., 2009, “Virus free induction of pluripotency and subsequent excision of reprogramming factors,” Nature 458:771-775; Woltjen et al., 2009, “piggyBac transposition reprograms fibroblasts to induced pluripotent stem cells,” Nature 458:766-770; Okita et al., 2008, “Generation of Mouse Induced Pluripotent Stem Cells Without Viral Vectors,” Science 322 (5903): 949-953; Stadtfeld et al., 2008, “Induced Pluripotent Stem Cells Generated without Viral Integration,” Science 322 (5903): 945-949; and Zhou et al., 2009, “Generation of Induced Pluripotent Stem Cells Using Recombinant Proteins,” Cell Stem Cell 4 (5): 381-384; each of which is hereby incorporated herein in its entirety.

In some aspects, exemplary iPS cell lines include but not limited to iPS-DF19-9; iPS-DF19-9; iPS-DF4-3; iPS-DF6-9; iPS (Foreskin); iPS (IMR90); and iPS (IMR90).

More details on the functions of signaling pathways relating to DE development can be found in, for example, Zorn and Wells, 2009, “Vertebrate endoderm development and organ formation,” Annu Rev Cell Dev Biol 25:221-251; Dessimoz et al., 2006, “FGF signaling is necessary for establishing gut tube domains along the anterior-posterior axis in vivo,” Mech Dev 123:42-55; McLin et al., 2007, “Repression of Wnt/B-catenin signaling in the anterior endoderm is essential for liver and pancreas development. Development,” 134:2207-2217; Wells and Melton, 2000, Development 127:1563-1572; de Santa Barbara et al., 2003, “Development and differentiation of the intestinal epithelium,” Cell Mol Life Sci 60 (7): 1322-1332; each of which is hereby incorporated herein in its entirety.

Any methods for producing definitive endoderm from pluripotent cells (e.g., iPSCs or ESCs) are applicable to the methods described herein. In some aspects, pluripotent cells are derived from a morula. In some aspects, pluripotent stem cells are stem cells. Stem cells used in these methods can include, but are not limited to, embryonic stem cells. Embryonic stem cells can be derived from the embryonic inner cell mass or from the embryonic gonadal ridges. Embryonic stem cells or germ cells can originate from a variety of animal species including, but not limited to, various mammalian species including humans. In some aspects, human embryonic stem cells are used to produce definitive endoderm. In some aspects, human embryonic germ cells are used to produce definitive endoderm. In some aspects, iPSCs are used to produce definitive endoderm.

In one aspect, the definitive endoderm may be derived from a precursor cell selected from an embryonic stem cell, an embryonic germ cell, an induced pluripotent stem cell, a mesoderm cell, a definitive endoderm cell, a posterior endoderm cell, a posterior endoderm cell, and a hindgut cell. In one aspect, the definitive endoderm may be derived from a pluripotent stem cell. In one aspect, the definitive endoderm may be derived from a pluripotent stem cell selected from an embryonic stem cell, an adult stem cell, or an induced pluripotent stem cell. In one aspect, the DE may be a DE monolayer, wherein greater than 90% of the cells in the DE monolayer co-express FOXA2 and SOX17.

In one aspect, the definitive endoderm may be derived from contacting a pluripotent stem cell with one or more molecules selected from Activin, the BMP subgroups of the TGF-beta superfamily of growth factors; Nodal, Activin A, Activin B, BMP4, Wnt3a, and combinations thereof.

In one aspect, the BMP signaling pathway inhibitor may be selected from Noggin, Dorsomorphin, LDN193189, DMH-1, and combinations thereof. In one aspect, the BMP signaling pathway inhibitor is Noggin. The BMP inhibitor may be present at a concentration of between from about 50 to about 1500 ng/ml.

In one aspect, the WNT signaling pathway activator may be selected from one or more molecules selected from the group consisting of Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, Wnt16, a GSKB inhibitor (e.g., CHIR99021, i.e. “CHIRON”), BIO, LY2090314, SB-216763, lithium, SFRP inhibitor WAY-316606, beta-catenin activator DCA. The concentration of the Wnt pathway activator may be, for example, used at a concentration between about 50 to about 1500 ng/ml. There are many ways to activate the Wnt/beta-catenin pathway (see http://web.stanford.edu/group/nusselab/cgi-bin/wnt/). Suitable Some existing wnt signaling pathway activators include but are not limited to protein-based activators, which may include Wnt ligands including but not limited to Wnt1, Wnt2, Wnt2b, Wnt3, Wnt3a, Wnt8, et al; modifiers of Wnt ligand activity including but not limited to activated Wnt frizzled receptors, (LRP) co-receptors, R-spondin proteins, Dkk proteins, regulators of Wnt ligand secretion and trafficking (Wntless, Porcupine), inhibiting beta-catenin degredation APC and GSK3beta inhibition, activated beta-catenin, constitutively active TCF/Lef proteins and chemical activators, which may include over 28 known chemicals that either activate or inhibit Wnt/beta-catenin signaling. Some activators include but are not limited to GSK3-beta inhibitors CHIR99021 (CHIRON), BIO, LY2090314, SB-216763, lithium, SFRP inhibitor WAY-316606, beta-catenin activator DCA.

In one aspect, the FGF signaling pathway activator may be one or more molecules selected from the group consisting of FGF1, FGF2, FGF3, FGF4, FGF10, FGF11, FGF12, FGF13, FGF14, FGF15, FGF16, FGF17, FGF18, FGF19, FGF20, FGF21, FGF22, FGF23, and combinations thereof, preferably FGF4 or FGF10, or a combination thereof. In one aspect, the concentration of the FGF pathway activator may be used at a concentration between about 50 to about 1500 ng/ml, though it will be understood by one of ordinary skill in the art that various concentrations may be used. Proteins and chemicals that stimulate the FGF receptor and signaling components downstream of the receptors including MAPK, MEK, ERK proteins and chemicals that modulate their activity. FGF signaling can be activated by inhibiting inhibitors of FGF signaling pathways including but not limited to Sprouty protein family members.

### Examples

The following non-limiting examples are provided to further illustrate embodiments of the invention disclosed herein. It should be appreciated by those of skill in the art that the techniques disclosed in the examples that follow represent approaches that have been found to function well in the practice of the invention, and thus may be considered to constitute examples of modes for its practice. However, those of skill in the art should, in light of the present disclosure, appreciate that many changes may be made in the specific embodiments that are disclosed and still obtain a like or similar result without departing from the spirit and scope of the invention.

Organogenesis is a complex and inter-connected process, orchestrated by multiple boundary tissue interactions¹⁻⁷. However, it is currently unclear how individual, neighboring components coordinate to establish an integral multi-organ structure. Disclosed herein is the continuous patterning and dynamic morphogenesis of hepatic, biliary and pancreatic structures, invaginating from a three-dimensional culture of human pluripotent stem cell (PSC).

Applicant has found that the boundary interactions between anterior and posterior gut spheroids differentiated from human PSCs enables autonomous emergence of hepato-biliary-pancreatic (HBP) organ domains specified at the foregut-midgut boundary organoids in the absence of extrinsic factor supply. Whereas transplant-derived tissues were dominated by midgut derivatives, long-term culture of micro dissected HBP organoids develop into a segregated hepato-pancreato-biliary anlage, followed by the recapitulation of early morphogenetic events including the invagination and branching of three different and inter-connected organ structures, reminiscent of tissues derived from mouse explanted foregut-midgut culture. Mis-segregation of multi-organ domains incurred by a genetic mutation in HES1 abolishes the biliary specification potential in culture, as seen in vivo^{8,9}. Applicant has demonstrated that the experimental multi-organ integrated model can be established by the juxta-positioning of foregut and midgut tissues, and which may serve as a tractable, manipulatable and easily accessible model for the study of complicated endoderm organogenesis in human.

The hepato-biliary-pancreatic (HBP) anlage, which is demarcated by HHEX (Hematopoietically-expressed homeobox protein) and PDX1 (Pancreatic and duodenal homeobox 1) expression is first specified at the boundary between the foregut-midgut¹⁰, and forms an epithelial vesicle invaginating ventrally from the primitive gut¹¹⁻¹³. The disruption of boundary-defining genes around this area such as BMPR1A (Bone morphogenetic protein receptor, type 1A)¹, HLX (H2.0-like homeobox)², CDX2 (Caudal type homeobox 2)³, NKX3-2 (NK3 Homcobox 2)⁴, HHEX⁵, PDX1 and SOX9 (SRY-box 9)⁶ significantly alters balanced organogenesis along the stomach-HBP-intestine in vivo¹⁻⁷. Subsequent diversification of HBP lineages is likely mediated by adjacent mesenchymal BMP at their boundary by indirectly repressing SOX9 in the posterior liver bud cells¹⁴. Thus, contiguous, dynamic organogenesis occurs in a complex environment and is likely driven by successive neighboring tissue interactions^{5,6,15}. However, the patterning and balanced organogenesis of the HBP system has not been successfully modelled in tissue culture due to technical complexities, hindering detailed mechanistic studies^{16,17}.

Here, Applicant used a three-dimensional differentiation approach using human pluripotent stem cells (PSCs) to specify gut spheroids with distinct regional identities comprised of both endoderm and mesoderm. Applicant demonstrated that antero-posterior interactions recapitulate the foregut (marked by SOX2, SRY-Box 2) and the midgut (marked by CDX2) boundary in vitro, modeling the inter-coordinated specification and invagination of the human hepato-biliary-pancreatic system.

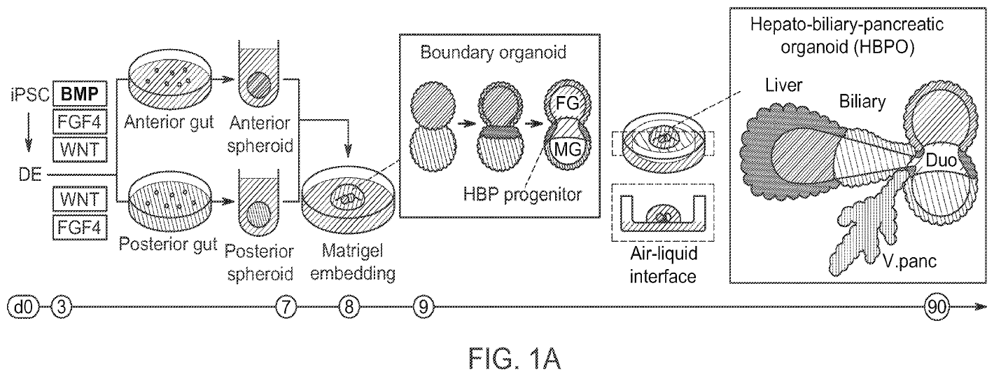

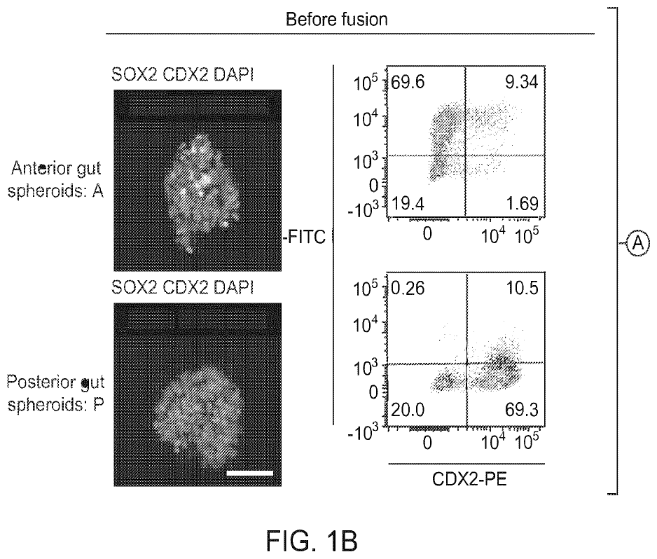

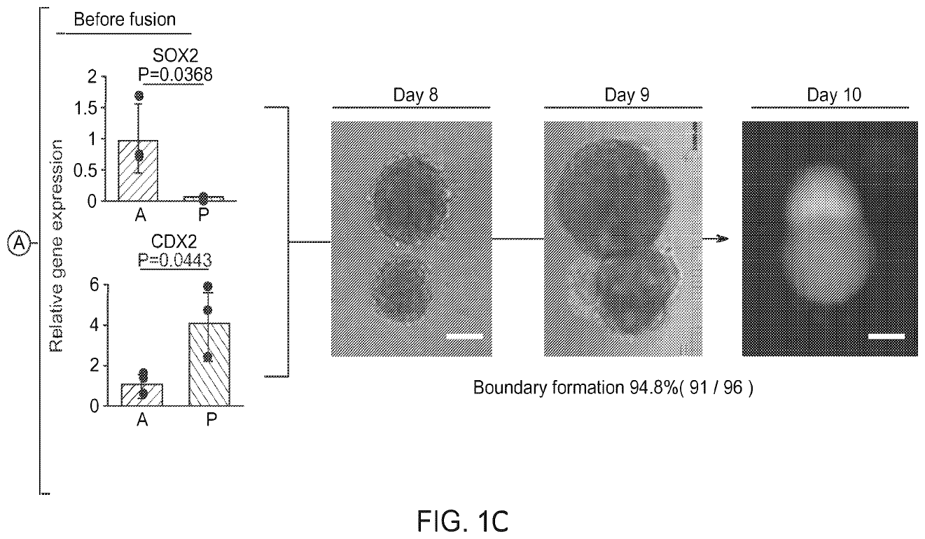

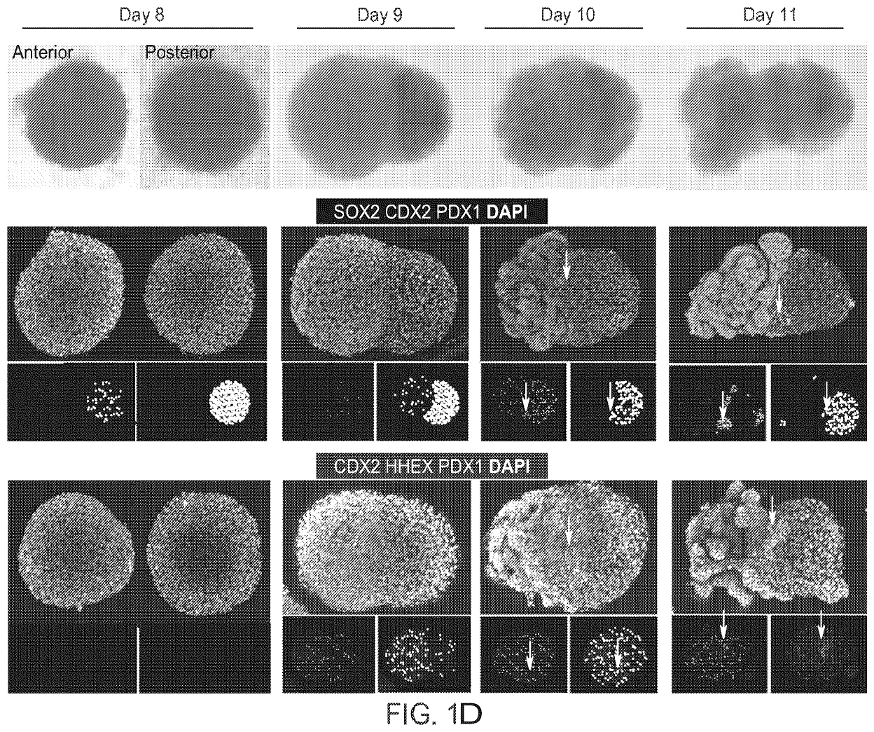

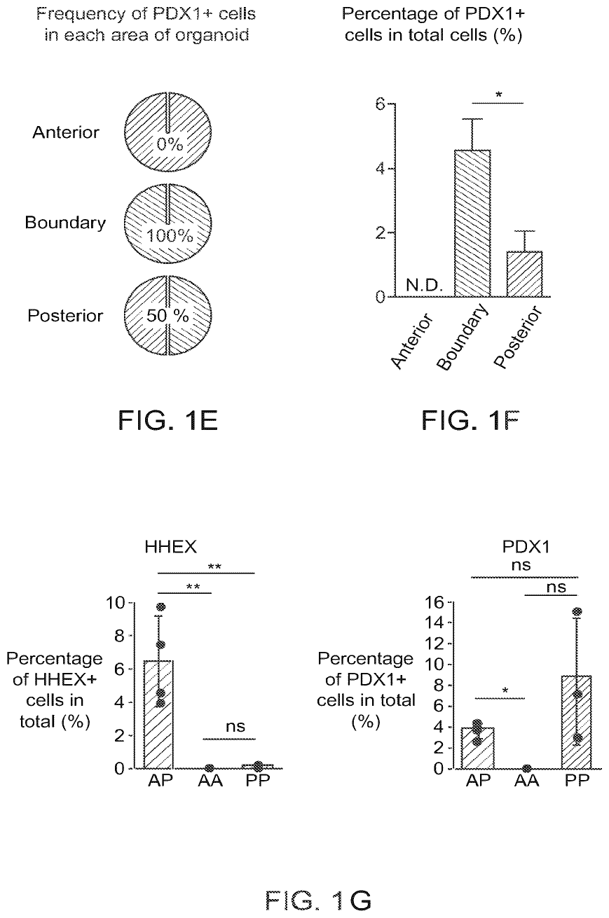

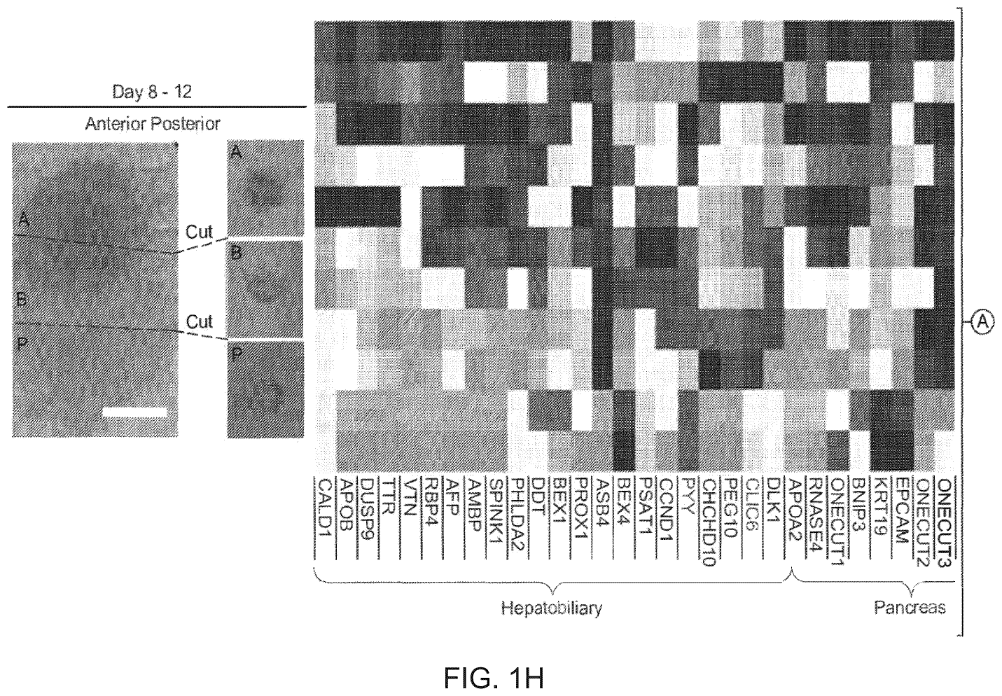

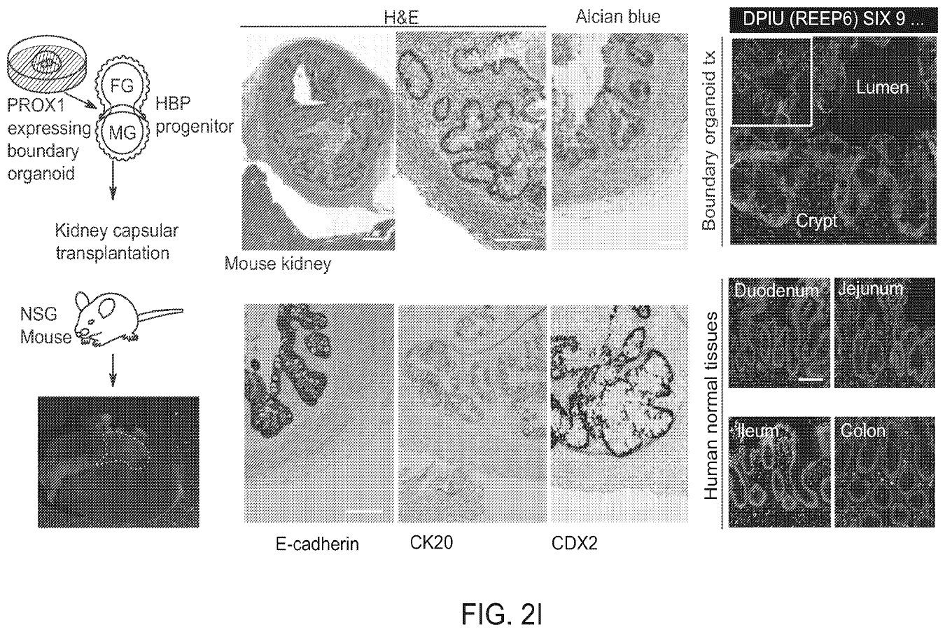

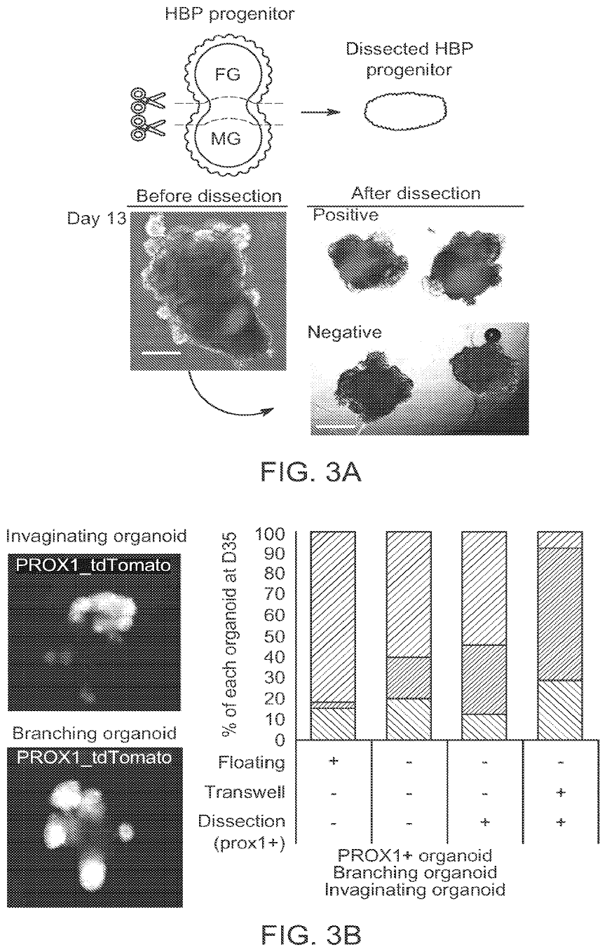

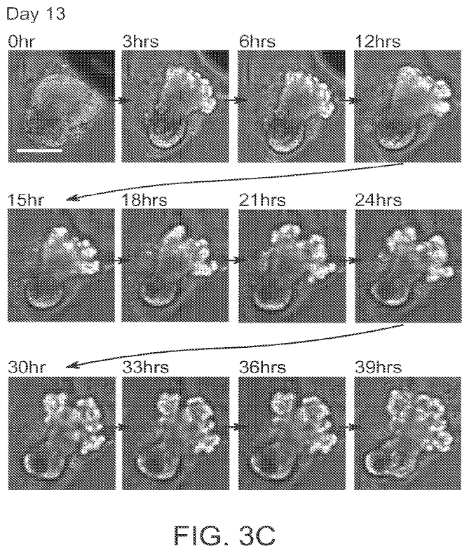

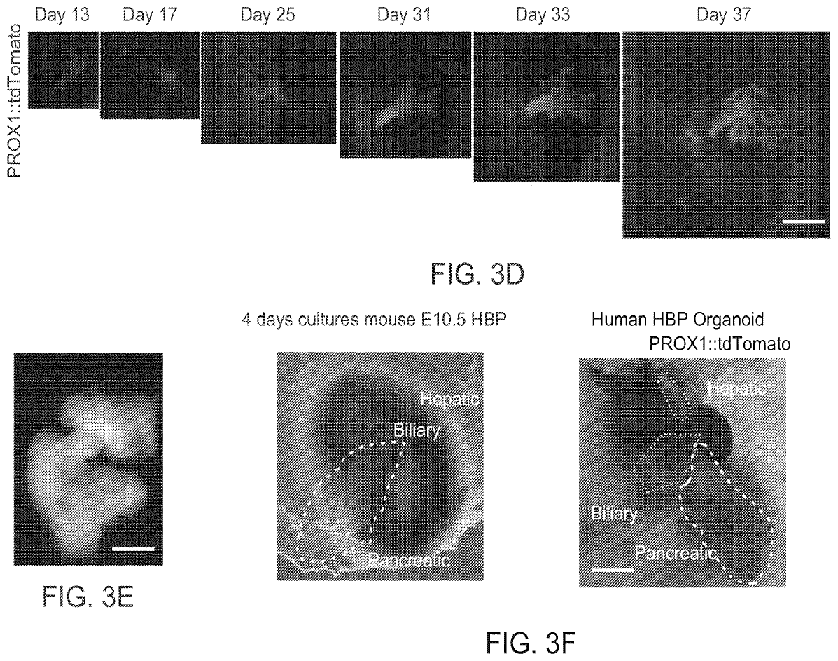

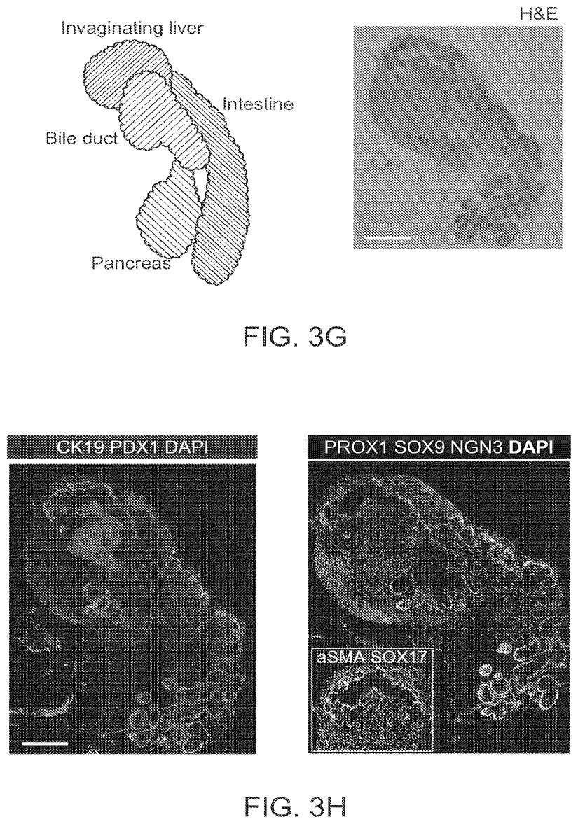

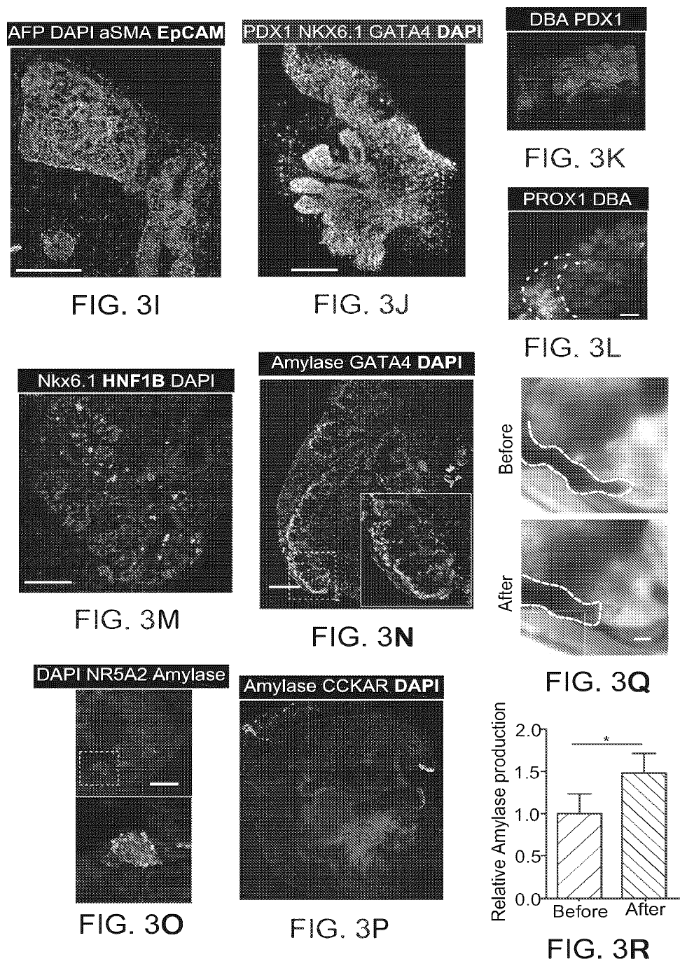

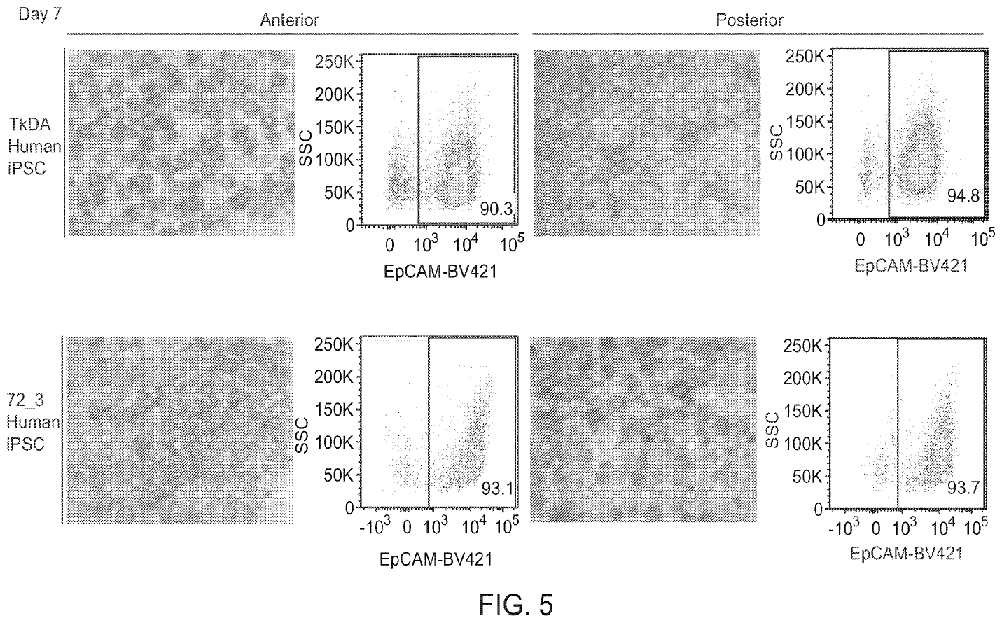

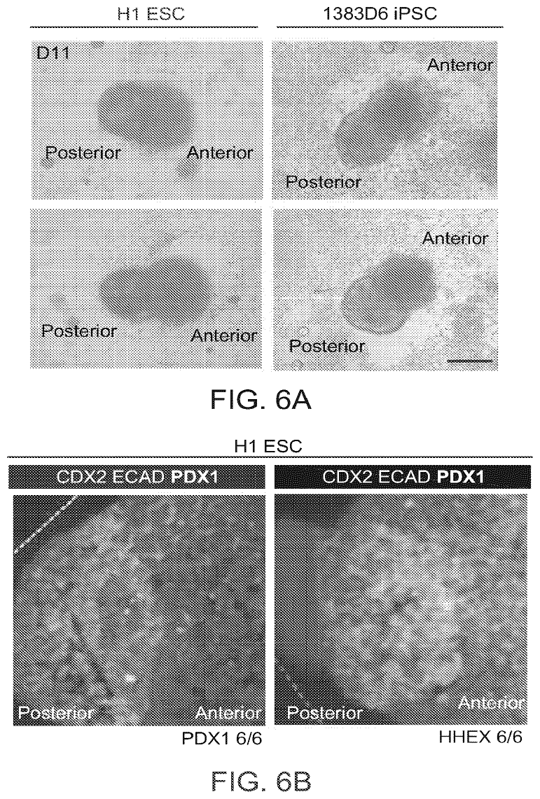

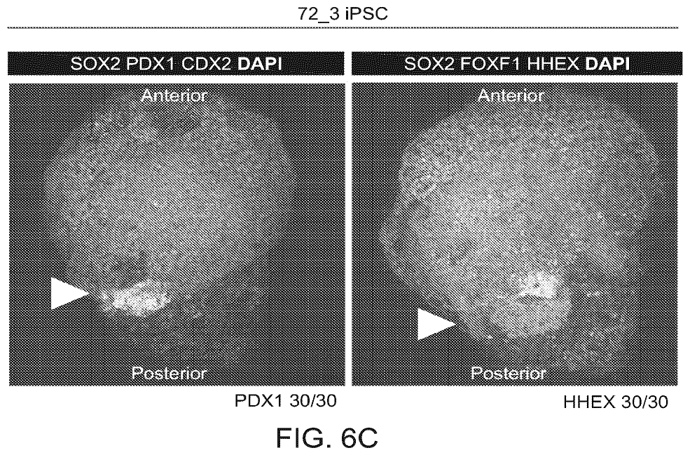

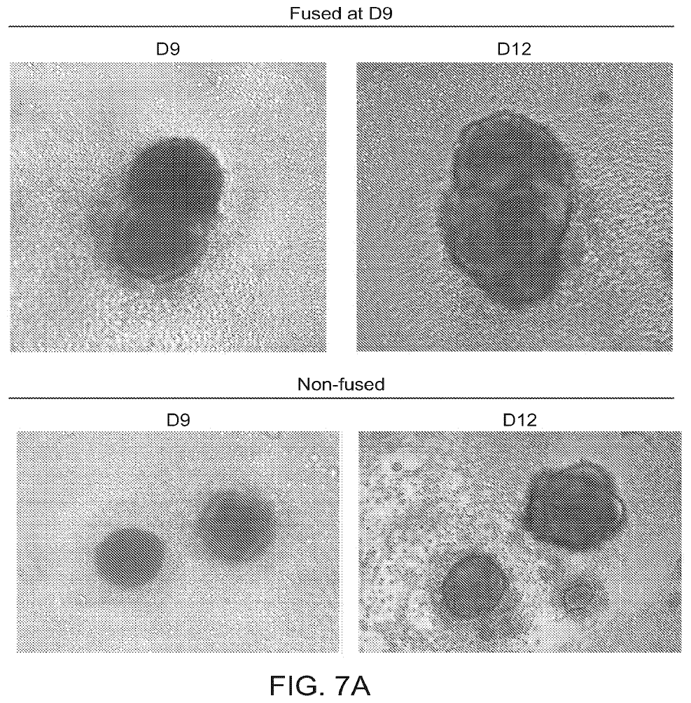

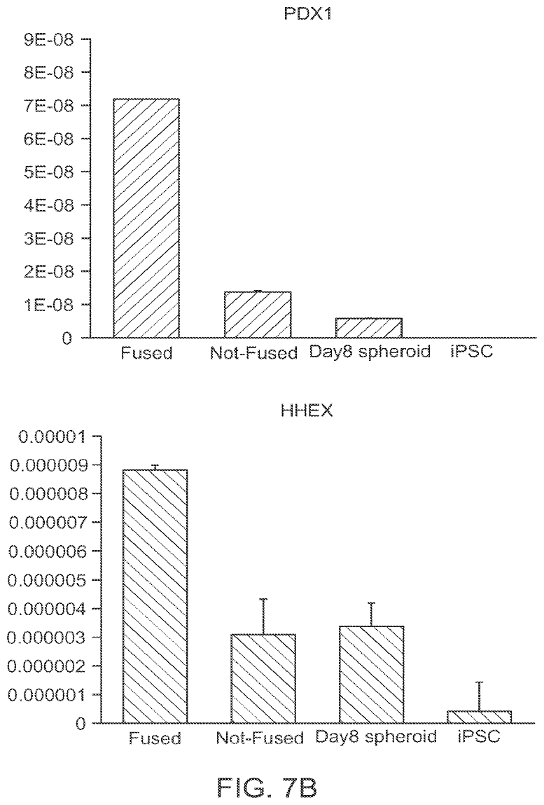

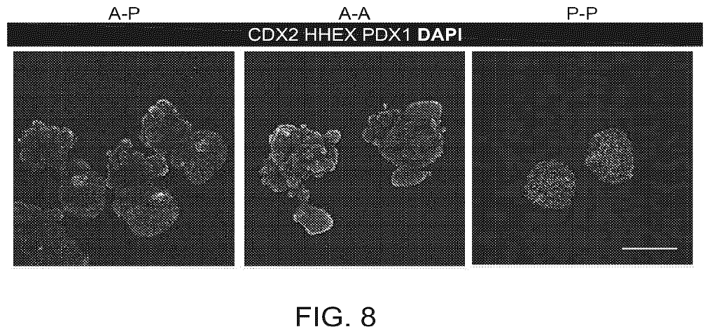

To develop a foregut-midgut boundary model, Applicant first exposed definitive endoderm cells, which were patterned as previously described 18.19, to the recombinant protein FGF4, and the small molecule CHIR99021 (that modulates the WNT pathway) in the presence of a BMP antagonist Noggin for anterior gut and in the absence of Noggin for posterior gut identity (FIG. 1A and FIG. 5). At Day 7 (D7), anterior or posterior gut cells were individually aggregated to form spheroids with a preferential expression of the foregut and mid/hindgut transcription factors SOX2 and CDX2, respectively (FIGS. 1B and 1C). Applicant then transferred a posterior gut spheroid adjacent to an anterior gut spheroid. At D9, the two spheroids were fused in 94.8% of the wells, and fused spheroids were embedded into a morphogenetic factor, namely Matrigel (FIGS. 1A and B and C). Surprisingly, without adding any exogenous factors, the HBP primordium emerged at the interface of the anterior and posterior gut spheroids, as evidenced by whole mount staining of the spheroids over D9, D10 and D11, using the markers SOX2, CDX2, the immature liver marker HHEX, and the antrum, duodenum and pancreas progenitor marker PDX1 (FIG. 1D). At D9, the anterior and posterior gut spheroids did not exhibit any expression of HHEX and PDX1. At D10, HHEX and PDX1 expressions were emerged, and HHEX was only detectable at the boundary of the anterior and posterior (bAP) organoids. Both PDX1 and HHEX positive cells were distinctively increased in the boundary region at D11 (FIG. 1D). The use of three additional induced pluripotent stem cells (iPSCs) and embryonic stem cells (ESCs) lines confirmed the reproducibility in developing HHEX and PDX1 expressing cells at the boundary of bAP organoids (FIG. 6A-6C). HBP progenitor induction requires cell-to-cell contact between the anterior and posterior gut spheroids (FIG. 3A and FIG. 3B). Of all the detected PDX1 positive cells at the boundary site (30 out of 30 stained organoids)(FIG. 1E), the percentage of PDX1 positive cells per total cell number was 5% in the boundary region while 0 and 1% in the anterior and the posterior region, respectively (FIG. 1F). PDX1 expressing cells were observed in A-P organoids and posterior-posterior (P-P) gut spheroid combinations. In contrast, HHEX positive cells were only detected in A-P organoids, but not in anterior-anterior (A-A) nor P-P combinations (FIG. 1G and FIG. 8), indicating the balanced induction of the HBP progenitors involves A-P fusion.

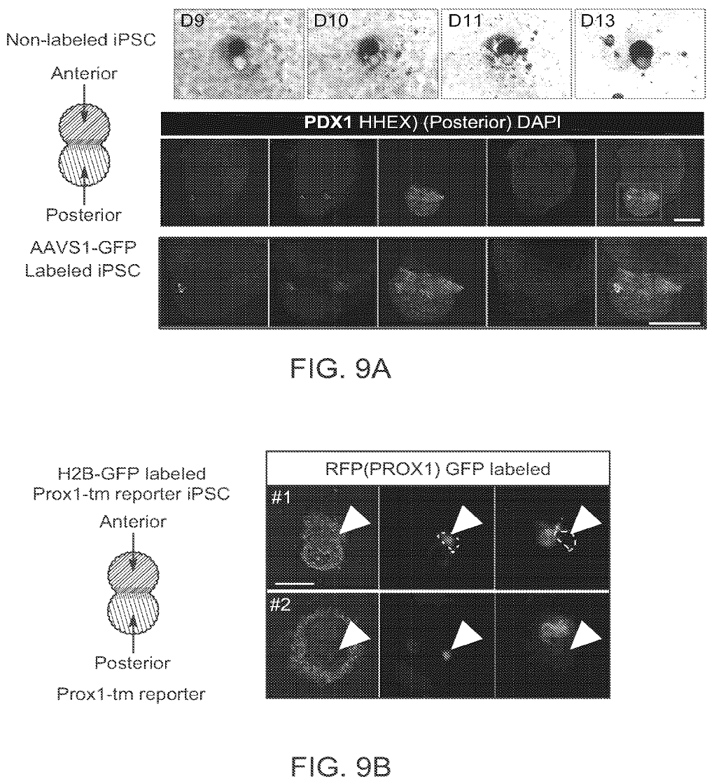

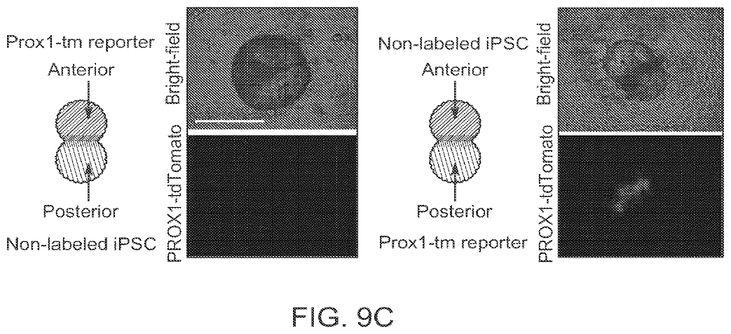

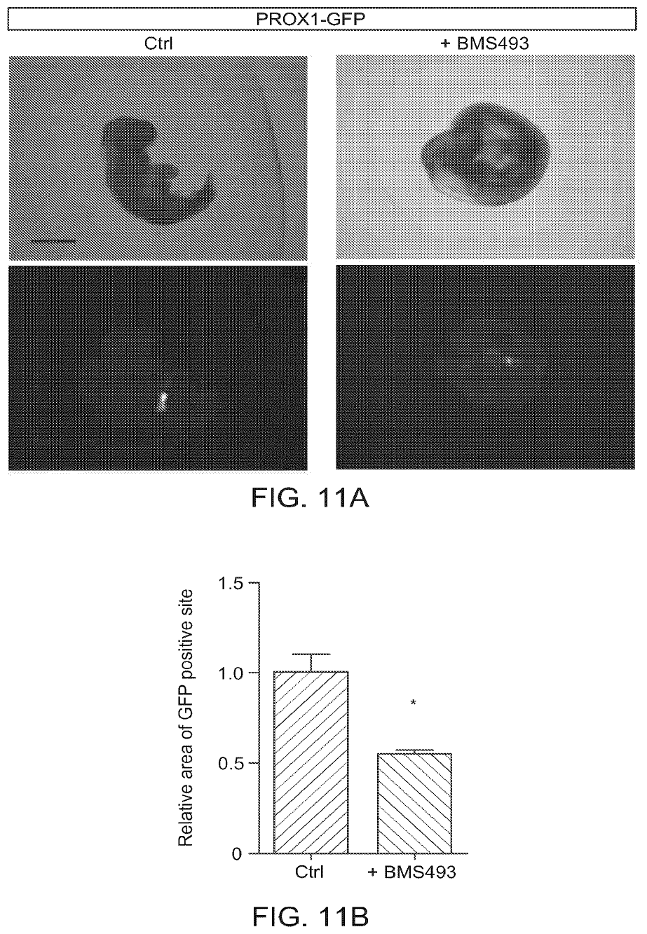

To trace the source of HHEX and PDX1 expressing cells, the bAP organoids were established using non-labeled anterior and GFP-labeled posterior gut spheroids. Applicant found that both HHEX and PDX1 expression overlapped with GFP, suggesting that the HBP progenitors originate from the posterior gut (FIG. 9A). RNA-sequencing of D8, D9, D11 and D12 micro-dissected anterior, boundary and posterior regions showed that the boundary tissues at D11 and D12 progressively expressed the arrays of HBP specification markers, whereas anterior or posterior regions gained foregut or mid/hindgut identity, respectively (FIGS. 1H and I). Of note, in agreement with A-P recombination experiments, posterior tissues possess a closer identity to boundary regions (FIGS. 11H and I). Taken together, the AP boundary strategy orchestrates autonomous patterning of HHEX and PDX1 positive HBP progenitors in the absence of exogenous inductive factors.

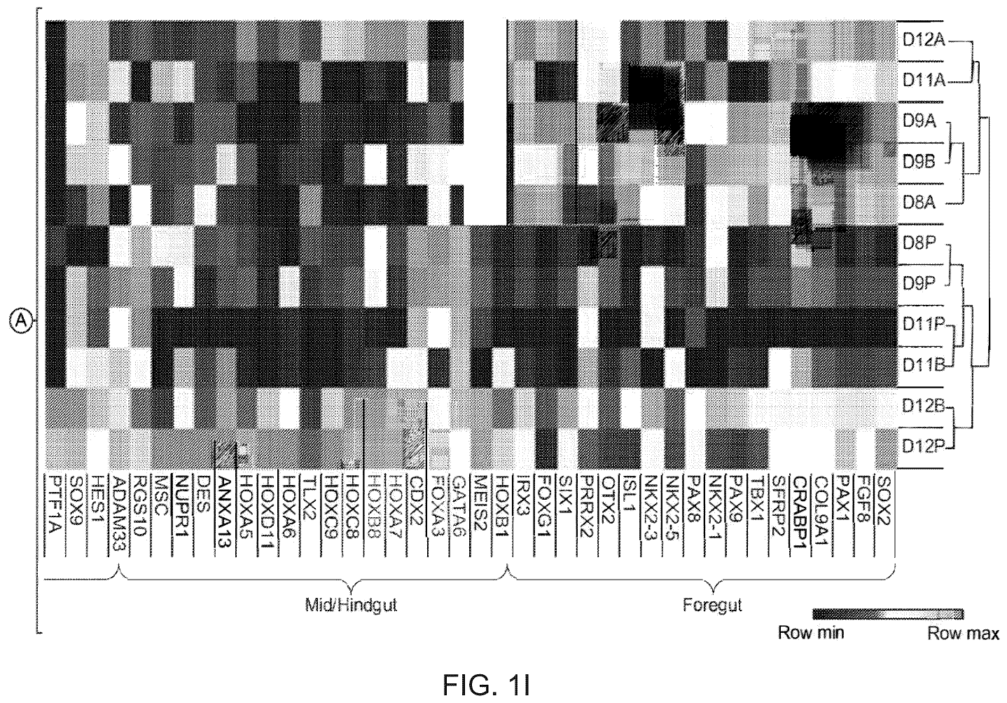

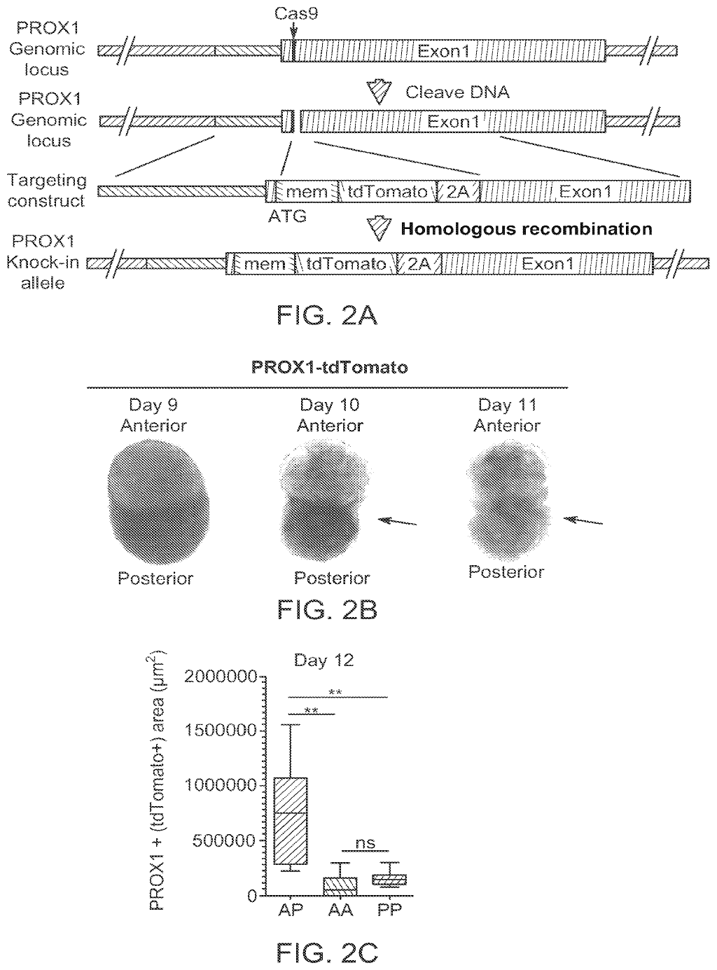

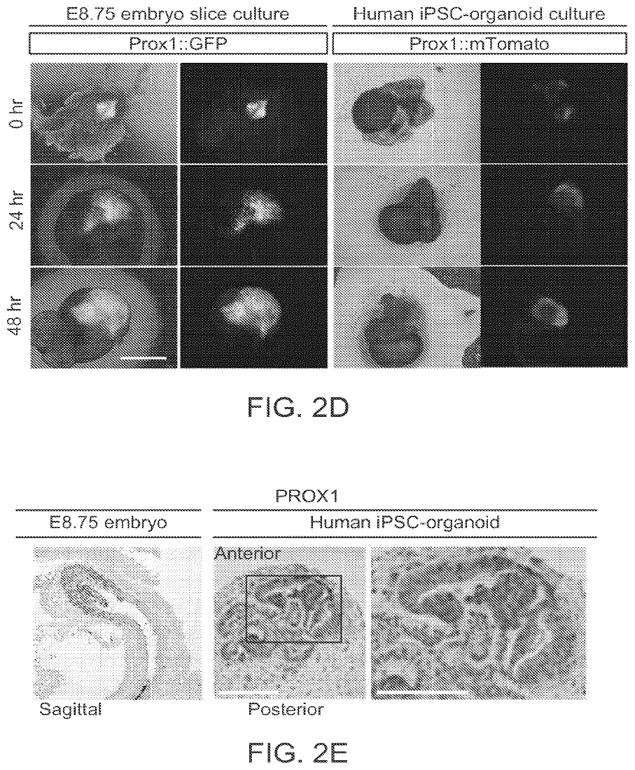

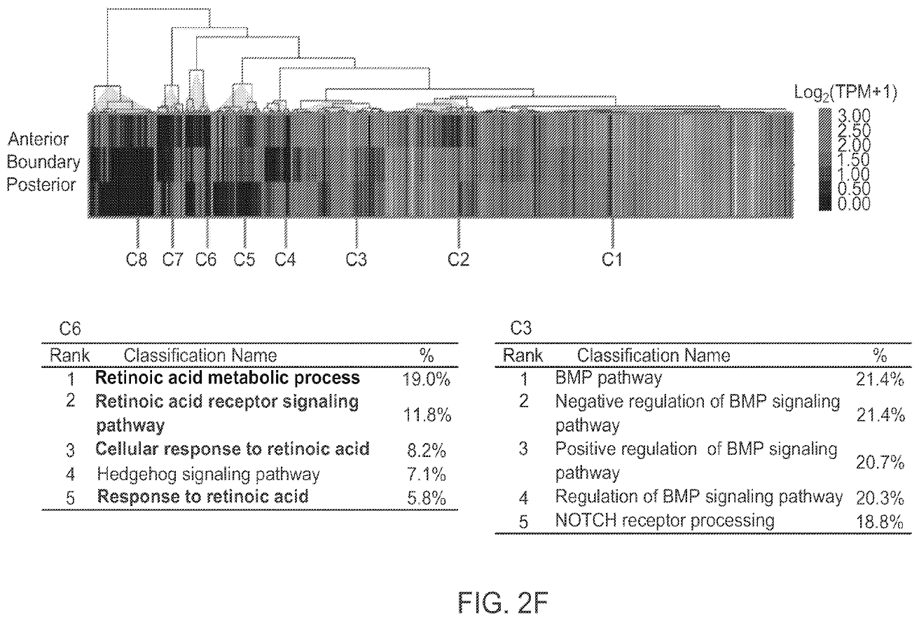

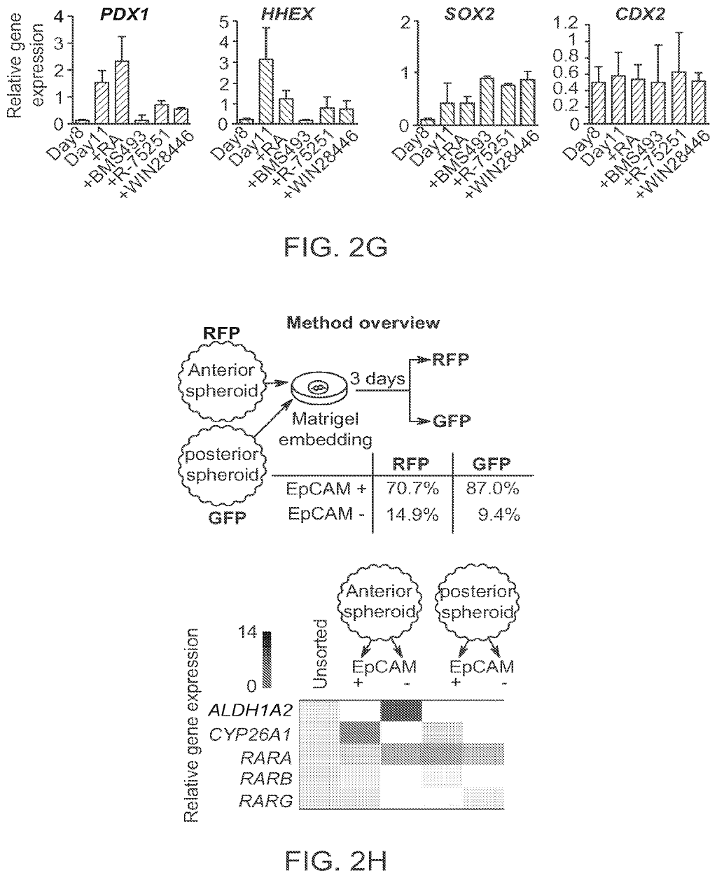

Applicant further established a reporter human iPSC to track their fate by visualizing tdTomato under the common progenitor marker of the liver, bile duct, and pancreas, a prospero-related homeobox 1 (PROX1) using CRISPR/Cas9 genome editing (FIG. 2A). Similar to HHEX and PDX1, PROX1 expression initiated at the boundary at D10 and increased afterwards (FIG. 2B). The PROX1 emergence was specific to the A-P organoids but not in A-A nor P-P combinations (FIG. 2C). A-P recombination assays indicated PROX1 positive cells also originated from the posterior gut cells (FIGS. 9B and 9C). Air-liquid interface culture induced additional growth of the PROX1 positive area, as seen in E8.75 Prox 1::EGFP mouse embryonic liver tissue (FIG. 2D). PROX1 immunostaining confirmed that the morphologically invaginating tissue induced from bAP organoids was similar with the boundary region of mouse at E8.5-8.75 (FIG. 2E).

To delineate the HBP progenitor self-inductive mechanism, Applicant evaluated the boundary specific expression profiles of known inductive signaling pathways. Amongst FGF. BMP. HH (hedgehog). NOTCH and RA (retinoic acid) signals. RNAseq identified that the signal downstream of RA were activated prominently at the boundary region, but not in the anterior or posterior regions at D11 (FIG. 2F and Table1).

TABLE 1
Gene ontology analysis for RNA sequencing (related to FIG. 2E)

| | | After | |
|---|---|---|---|
| | Orig- | restricting | |
| | inal | to | |
| Term | size | dataset | % |
| C1 | | | |
| BIOCARTA_TGFB_PATHWAY | 19 | 12 | 63.2% |
| PID_RETINOIC_ACID_PATHWAY | 30 | 17 | 56.7% |
| GO_NOTCH_RECEPTOR_PROCESSING | 16 | 9 | 56.3% |
| GO_POSITIVE_REGULATION_OF_ | 33 | 18 | 54.5% |
| NOTCH_SIGNALING_PATHWAY | | | |
| PID_FGF_PATHWAY | 55 | 29 | 52.7% |
| GO_POSITIVE_REGULATION_OF_ | 29 | 15 | 51.7% |
| BMP_SIGNALING_PATHWAY | | | |
| GO_REGULATION_OF_NOTCH_ | 63 | 32 | 50.8% |
| SIGNALING_PATHWAY | | | |
| GO_NOTCH_SIGNALING_PATHWAY | 111 | 50 | 45.0% |
| HALLMARK_HEDGEHOG_SIGNALING | 35 | 15 | 42.9% |
| PID_BMP_PATHWAY | 42 | 18 | 42.9% |
| GO_REGULATION_OF_BMP_ | 74 | 27 | 36.5% |
| SIGNALING_PATHWAY | | | |
| GO_NEGATIVE_REGULATION_OF_ | 25 | 9 | 36.0% |
| NOTCH_SIGNALING_PATHWAY | | | |
| KEGG_HEDGEHOG_SIGNALING_P | 56 | 19 | 33.9% |
| ATHWAY | | | |
| GO_RESPONSE_TO_RETINOIC_ACID | 103 | 33 | 32.0% |
| GO_NOTCH_BINDING | 17 | 5 | 29.4% |
| GO_REGULATION_OF_ | 29 | 8 | 27.6% |
| RETINOIC_ACID_ | | | |
| RECEPTOR_SIGNALING_PATHWAY | | | |
| GO_CELLULAR_RESPONSE_ | 61 | 15 | 24.6% |
| TO_RETINOIC_ACID | | | |
| GO_RETINOIC_ACID_ | 21 | 5 | 23.8% |
| METABOLIC_PROCESS | | | |
| GO_NEGATIVE_REGULATION_OF_ | 42 | 10 | 23.8% |
| BMP_SIGNALING_PATHWAY | | | |
| GO_RETINOIC_ACID_RECEPTOR_ | 17 | 4 | 23.5% |
| SIGNALING_PATHWAY | | | |
| GO_NEGATIVE_REGULATION_ | 23 | 3 | 13.0% |
| OF_RETINOIC_ACID_ | | | |
| RECEPTOR_SIGNALING_PATHWAY | | | |
| C2 | | | |
| GO_NEGATIVE_REGULATION_OF_ | 25 | 5 | 20.0% |
| NOTCH_SIGNALING_PATHWAY | | | |
| GO_NEGATIVE_REGULATION_OF_ | 42 | 8 | 19.0% |
| BMP_SIGNALIN_PATHWAY | | | |
| BIOCARTA_TGFB_PATHWAY | 19 | 3 | 15.8% |
| GO_RESPONSE_TO_RETINOIC_ACID | 103 | 16 | 15.5% |
| GO_REGULATION_OF_BMP_ | 74 | 11 | 14.9% |
| SIGNALING_PATHWAY | | | |
| PID_FGF_PATHWAY | 55 | 8 | 14.5% |
| GO_RETINOIC_ACID_METABOLIC_ | 21 | 3 | 14.3% |
| PROCESS | | | |
| GO_NOTCH_SIGNALING_PATHWAY | 111 | 15 | 13.5% |
| GO_NOTCH_RECEPTOR_PROCESSING | 16 | 2 | 12.5% |
| PID_BMP_PATHWAY | 42 | 5 | 11.9% |
| GO_RETINOIC_ACID_RECEPTOR_ | 17 | 2 | 11.8% |
| SIGNALING_PATHWAY | | | |
| GO_CELLULAR_RESPONSE_ | 61 | 7 | 11.5% |
| TO_RETINOIC_ACID | | | |
| HALLMARK_HEDGEHOG_SIGNALING | 35 | 4 | 11.4% |
| KEGG_HEDGEHOG_SIGNALING_ | 56 | 6 | 10.7% |
| PATHWAY | | | |
| GO_POSITIVE_REGULATION_ | 29 | 3 | 10.3% |
| OF_BMP_SIGNALING_PATHWAY | | | |
| GO_REGULATION_OF_NOTCH_ | 63 | 6 | 9.5% |
| SIGNALING_PATHWAY | | | |
| PID_RETINOIC_ACID_PATHWAY | 30 | 2 | 6.7% |
| GO_NOTCH_BINDING | 17 | 1 | 5.9% |
| GO_REGULATION_OF_RETINOIC_ | 29 | 1 | 3.4% |
| ACID_RECEPTOR_SIGNALING_ | | | |
| PATHWAY | | | |
| GO_POSITIVE_REGULATION_OF_ | 33 | 1 | 3.0% |
| NOTCH_SIGNALING_PATHWAY | | | |
| GO_NEGATIVE_REGULATION_ | 23 | | 0.0% |
| OF_RETINOIC_ACID_ | | | |
| RECEPTOR_SIGNALING_PATHWAY | | | |
| C3 | | | |
| PID_BMP_PATHWAY | 42 | 9 | 21.4% |
| GO_NEGATIVE_REGULATION_OF_ | 42 | 9 | 21.4% |
| BMP_SIGNALING_PATHWAY | | | |
| GO_POSITIVE_REGULATION_ | 29 | 6 | 20.7% |
| OF_BMP_SIGNALING_PATHWAY | | | |
| GO_REGULATION_OF_BMP_ | 74 | 15 | 20.3% |
| SIGNALING_PATHWAY | | | |
| GO_NOTCH_RECEPTOR_PROCESSING | 16 | 3 | 18.8% |
| GO_NOTCH_BINDING | 17 | 3 | 17.6% |
| PID_RETINOIC_ACID_PATHWAY | 30 | 5 | 16.7% |
| BIOCARTA_TGFB_PATHWAY | 19 | 3 | 15.8% |
| GO_CELLULAR_RESPONSE_ | 61 | 9 | 14.8% |
| TO_RETINOIC_ACID | | | |
| GO_RETINOIC_ACID_METABOLIC_ | 21 | 3 | 14.3% |
| PROCESS | | | |
| KEGG_HEDGEHOG_SIGNALING_ | 56 | 8 | 14.3% |
| PATHWAY | | | |
| GO_RESPONSE_TO_RETINOIC_ACID | 103 | 14 | 13.6% |
| GO_NEGATIVE_REGULATION_ | 23 | 3 | 13.0% |
| OF_RETINOIC_ACID_RECEPTOR_ | | | |
| SIGNALING_PATHWAY | | | |
| GO_POSITIVE_REGULATION_OF_ | 33 | 4 | 12.1% |
| NOTCH_SIGNALING_PATHWAY | | | |
| GO_NOTCH_SIGNALING_PATHWAY | 111 | 12 | 10.8% |
| GO_REGULATION_OF_ | 29 | 3 | 10.3% |
| RETINOIC_ACID_RECEPTOR_ | | | |
| SIGNALING_PATHWAY | | | |
| HALLMARK_HEDGEHOG_SIGNALING | 35 | 3 | 8.6% |
| GO_REGULATION_OF_NOTCH_ | 63 | 5 | 7.9% |
| SIGNALING_PATHWAY | | | |
| PID_FGF_PATHWAY | 55 | 4 | 7.3% |
| GO_RETINOIC_ACID_RECEPTOR_ | 17 | 1 | 5.9% |
| SIGNALING_PATHWAY | | | |
| GO_NEGATIVE_REGULATION_OF_ | 25 | 1 | 4.0% |
| NOTCH_SIGNALING_PATHWAY | | | |
| C4 | | | |
| GO_NEGATIVE_REGULATION_OF_ | 25 | 3 | 12.0% |
| NOTCH_SIGNALING_PATHWAY | | | |
| PID_RETINOIC_ACID_PATHWAY | 30 | 2 | 6.7% |
| GO_NOTCH_SIGNALING_PATHWAY | 111 | 7 | 6.3% |
| GO_NOTCH_BINDING | 17 | 1 | 5.9% |
| GO_RETINOIC_ACID_RECEPTOR_ | 17 | 1 | 5.9% |
| SIGNALING_PATHWAY | | | |
| GO_REGULATION_OF_NOTCH_ | 63 | 3 | 4.8% |
| SIGNALING_PATHWAY | | | |
| PID_FGF_PATHWAY | 55 | 2 | 3.6% |
| KEGG_HEDGEHOG_SIGNALING_ | 56 | 2 | 3.6% |
| PATHWAY | | | |
| GO_CELLULAR_RESPONSE_ | 61 | 2 | 3.3% |
| TO_RETINOIC_ACID | | | |
| GO_POSITIVE_REGULATION_OF_ | 33 | 1 | 3.0% |
| NOTCH_SIGNALING_PATHWAY | | | |
| GO_RESPONSE_TO_RETINOIC_ACID | 103 | 3 | 2.9% |
| PID_BMP_PATHWAY | 42 | 1 | 2.4% |
| GO_NEGATIVE_REGULATION_OF_ | 42 | 1 | 2.4% |
| BMP_SIGNALING_PATHWAY | | | |
| GO_REGULATION_OF_BMP_ | 74 | 1 | 1.4% |
| SIGNALING_PATHWAY | | | |
| GO_NOTCH_RECEPTOR_PROCESSING | 16 | | 0.0% |
| GO_RETINOIC_ACID_ | 21 | | 0.0% |
| METABOLIC_PROCESS | | | |
| BIOCARTA_TGFB_PATHWAY | 19 | | 0.0% |
| HALLMARK_HEDGEHOG_SIGNALING | 35 | | 0.0% |
| GO_POSITIVE_REGULATION_ | 29 | | 0.0% |
| OF_BMP_SIGNALING_PATHWAY | | | |
| GO_REGULATION_OF_ | 29 | | 0.0% |
| RETINOIC_ACID_RECEPTOR_ | | | |
| SIGNALING_PATHWAY | | | |
| GO_NEGATIVE_REGULATION_ | 23 | | 0.0% |
| OF_RETINOIC_ACID_ | | | |
| RECEPTOR_SIGNALING_PATHWAY | | | |
| C5 | | | |
| HALLMARK_HEDGEHOG_SIGNALING | 35 | 6 | 17.1% |
| PID_BMP_PATHWAY | 42 | 5 | 11.9% |
| GO_NOTCH_BINDING | 17 | 2 | 11.8% |
| GO_RETINOIC_ACID_RECEPTOR_ | 17 | 2 | 11.8% |
| SIGNALING_PATHWAY | | | |
| GO_CELLULAR_RESPONSE_ | 61 | 6 | 9.8% |
| TO_RETINOIC_ACID | | | |
| GO_RETINOIC_ACID_ | 21 | 2 | 9.5% |
| METABOLIC_PROCESS | | | |
| GO_NEGATIVE_REGULATION_OF_ | 42 | 4 | 9.5% |
| BMP_SIGNALING_PATHWAY | | | |
| KEGG_HEDGEHOG_SIGNALING_ | 56 | 5 | 8.9% |
| PATHWAY | | | |
| GO_RESPONSE_TO_RETINOIC_ACID | 103 | 8 | 7.8% |
| GO_REGULATION_OF_BMP_ | 74 | 5 | 6.8% |
| SIGNALING_PATHWAY | | | |
| GO_NOTCH_SIGNALING_PATHWAY | 111 | 7 | 6.3% |
| PID_FGF_PATHWAY | 55 | 3 | 5.5% |
| BIOCARTA_TGFB_PATHWAY | 19 | 1 | 5.3% |
| GO_NEGATIVE_REGULATION_ | 23 | 1 | 4.3% |
| OF_RETINOIC_ACID_ | | | |
| RECEPTOR_SIGNALING_PATHWAY | | | |
| GO_NEGATIVE_REGULATION_OF_ | 25 | 1 | 4.0% |
| NOTCH_SIGNALING_PATHWAY | | | |
| GO_POSITIVE_REGULATION_OF_ | 29 | 1 | 3.4% |
| BMP_SIGNALING_PATHWAY | | | |
| GO_REGULATION_OF_ | 29 | 1 | 3.4% |
| RETINOIC_ACID_RECEPTOR_ | | | |
| SIGNALING_PATHWAY | | | |
| PID_RETINOIC_ACID_PATHWAY | 30 | 1 | 3.3% |
| GO_REGULATION_OF_NOTCH_ | 63 | 2 | 3.2% |
| SIGNALING_PATHWAY | | | |
| GO_NOTCH_RECEPTOR_PROCESSING | 16 | | 0.0% |
| GO_POSITIVE_REGULATION_OF_ | 33 | | 0.0% |
| NOTCH_SIGNALING_PATHWAY | | | |
| C6 | | | |
| GO_RETINOIC_ACID_ | 21 | 4 | 19.0% |
| METABOLIC_PROCESS | | | |
| GO_RETINOIC_ACID_RECEPTOR_ | 17 | 2 | 11.8% |
| SIGNALING_PATHWAY | | | |
| GO_CELLULAR_RESPONSE_ | 61 | 5 | 8.2% |
| TO_RETINOIC_ACID | | | |
| KEGG_HEDGEHOG_SIGNALING_P | 56 | 4 | 7.1% |
| ATHWAY | | | |
| GO_RESPONSE_TO_RETINOIC_ACID | 103 | 6 | 5.8% |
| HALLMARK_HEDGEHOG_SIGNALING | 35 | 2 | 5.7% |
| GO_NEGATIVE_REGULATION_ | 23 | 1 | 4.3% |
| OF_RETINOIC_ACID_ | | | |
| RECEPTOR_SIGNALING_PATHWAY | | | |
| GO_NEGATIVE_REGULATION_OF_ | 25 | 1 | 4.0% |
| NOTCH_SIGNALING_PATHWAY | | | |
| GO_REGULATION_OF_ | 29 | 1 | 3.4% |
| RETINOIC_ACID_RECEPTOR_ | | | |
| SIGNALING_PATHWAY | | | |
| PID_RETINOIC_ACID_PATHWAY | 30 | 1 | 3.3% |
| GO_NOTCH_SIGNALING_PATHWAY | 111 | 3 | 2.7% |
| GO_REGULATION_OF_NOTCH_ | 63 | 1 | 1.6% |
| SIGNALING_PATHWAY | | | |
| GO_NOTCH_RECEPTOR_PROCESSING | 16 | | 0.0% |
| GO_NOTCH_BINDING | 17 | | 0.0% |
| BIOCARTA_TGFB_PATHWAY | 19 | | 0.0% |
| GO_POSITIVE_REGULATION_OF_ | 33 | | 0.0% |
| NOTCH_SIGNALING_PATHWAY | | | |
| PID_BMP_PATHWAY | 42 | | 0.0% |
| PID_FGF_PATHWAY | 55 | | 0.0% |
| GO_POSITIVE_REGULATION_OF_ | 29 | | 0.0% |
| BMP_SIGNALING_PATHWAY | | | |
| GO_NEGATIVE_REGULATION_OF_ | 42 | | 0.0% |
| BMP_SIGNALING_PATHWAY | | | |
| GO_REGULATION_OF_BMP_ | 74 | | 0.0% |
| SIGNALING_PATHWAY | | | |
| C7 | | | |
| GO_CELLULAR_RESPONSE_ | 61 | 5 | 8.2% |
| TO_RETINOIC_ACID | | | |
| GO_NOTCH_RECEPTOR_PROCESSING | 16 | 1 | 6.3% |
| GO_POSITIVE_REGULATION_OF_ | 33 | 2 | 6.1% |
| NOTCH_SIGNALING_PATHWAY | | | |
| GO_NOTCH_BINDING | 17 | 1 | 5.9% |
| GO_RESPONSE_TO_RETINOIC_ACID | 103 | 6 | 5.8% |
| GO_REGULATION_OF_BMP_ | 74 | 4 | 5.4% |
| SIGNALING_PATHWAY | | | |
| KEGG_HEDGEHOG_ | 56 | 3 | 5.4% |
| SIGNALING_PATHWAY | | | |
| GO_RETINOIC_ACID_ | 21 | 1 | 4.8% |
| METABOLIC_PROCESS | | | |
| GO_REGULATION_OF_NOTCH_ | 63 | 3 | 4.8% |
| SIGNALING_PATHWAY | | | |
| GO_NEGATIVE_REGULATION_OF_ | 42 | 2 | 4.8% |
| BMP_SIGNALING_PATHWAY | | | |
| GO_NEGATIVE_REGULATION_OF_ | 25 | 1 | 4.0% |
| NOTCH_SIGNALING_PATHWAY | | | |
| GO_NOTCH_SIGNALING_PATHWAY | 111 | 4 | 3.6% |
| GO_POSITIVE_REGULATION_OF_ | 29 | 1 | 3.4% |
| BMP_SIGNALING_PATHWAY | | | |
| PID_BMP_PATHWAY | 42 | 1 | 2.4% |
| PID_FGF_PATHWAY | 55 | 1 | 1.8% |
| GO_RETINOIC_ACID_RECEPTOR_ | 17 | | 0.0% |
| SIGNALING_PATHWAY | | | |
| BIOCARTA_TGFB_PATHWAY | 19 | | 0.0% |
| HALLMARK_HEDGEHOG_SIGNALING | 35 | | 0.0% |
| PID_RETINOIC_ACID_PATHWAY | 30 | | 0.0% |
| GO_REGULATION_OF_ | 29 | | 0.0% |
| RETINOIC_ACID_RECEPTOR_ | | | |
| SIGNALING_PATHWAY | | | |
| GO_NEGATIVE_REGULATION_ | 23 | | 0.0% |
| OF_RETINOIC_ACID_ | | | |
| RECEPTOR_SIGNALING_PATHWAY | | | |
| C8 | | | |
| GO_NOTCH_BINDING | 17 | 3 | 17.6% |
| GO_CELLULAR_RESPONSE_ | 61 | 10 | 16.4% |
| TO_RETINOIC_ACID | | | |
| GO_POSITIVE_REGULATION_OF_ | 33 | 5 | 15.2% |
| NOTCH_SIGNALING_PATHWAY | | | |
| KEGG_HEDGEHOG_SIGNALING_ | 56 | 8 | 14.3% |
| PATHWAY | | | |
| GO_RESPONSE_TO_RETINOIC_ACID | 103 | 14 | 13.6% |
| GO_REGULATION_OF_NOTCH_ | 63 | 8 | 12.7% |
| SIGNALING_PATHWAY | | | |
| GO_NEGATIVE_REGULATION_OF_ | 25 | 3 | 12.0% |
| NOTCH_SIGNALING_PATHWAY | | | |
| HALLMARK_HEDGEHOG_SIGNALING | 35 | 4 | 11.4% |
| PID_FGF_PATHWAY | 55 | 6 | 10.9% |
| PID_RETINOIC_ACID_PATHWAY | 30 | 2 | 6.7% |
| GO_NOTCH_SIGNALING_PATHWAY | 111 | 7 | 6.3% |
| GO_NOTCH_RECEPTOR_PROCESSING | 16 | 1 | 6.3% |
| GO_RETINOIC_ACID_ | 21 | 1 | 4.8% |
| METABOLIC_PROCESS | | | |
| PID_BMP_PATHWAY | 42 | 2 | 4.8% |
| GO_NEGATIVE_REGULATION_OF_ | 42 | 2 | 4.8% |
| BMP_SIGNALING_PATHWAY | | | |
| GO_NEGATIVE_REGULATION_ | 23 | 1 | 4.3% |
| OF_RETINOIC_ACID_ | | | |
| RECEPTOR_SIGNALING_PATHWAY | | | |
| GO_POSITIVE_REGULATION_ | 29 | 1 | 3.4% |
| OF_BMP_SIGNALING_PATHWAY | | | |
| GO_REGULATION_OF_ | 29 | 1 | 3.4% |
| RETINOIC_ACID_RECEPTOR_ | | | |
| SIGNALING_PATHWAY | | | |
| GO_REGULATION_OF_BMP_ | 74 | 2 | 2.7% |
| SIGNALING_PATHWAY | | | |
| GO_RETINOIC_ACID_RECEPTOR_ | 17 | | 0.0% |
| SIGNALING_PATHWAY | | | |
| BIOCARTA_TGFB_PATHWAY | 19 | | 0.0% |

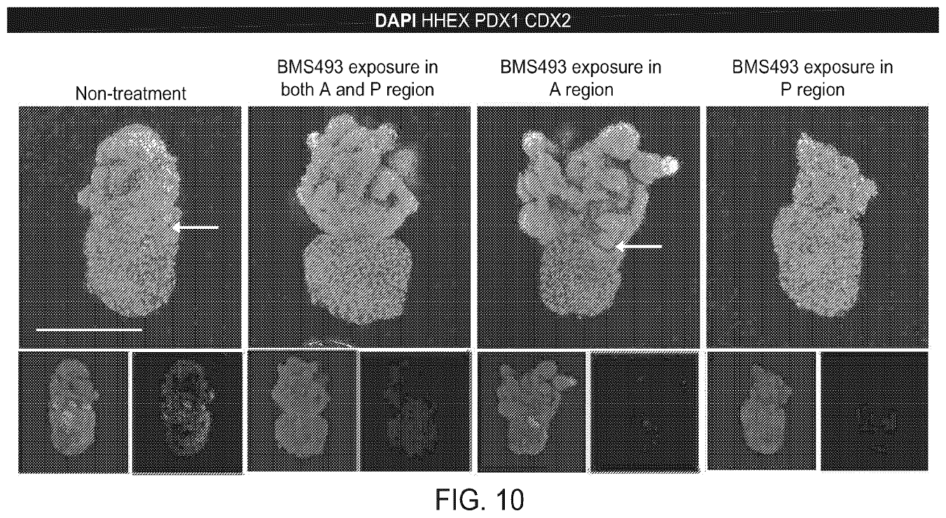

In support, Applicant exposed the bAP organoids at D9 to various RA signaling agonists or antagonists. The RA receptor antagonist BMS493 strongly suppressed the gene expression of both HHEX and PDX1 (FIG. 2G). Animal studies suggested that RA signaling has an important role in the lineage specification into the hepato-biliary-pancreatic systems^{20,21}. Lateral plate mesoderm population acts as an activator for RA signaling during the specification in vivo²²⁻²⁴. To implicate the cellular source for RA in the model system, RA signaling related genes were assessed in the isolated epithelial and non-epithelial cell populations. FACS analysis showed that there were 90.3% Epithelial Cell Adhesion Molecule (EpCAM) positive epithelial cells in the anterior gut cells, whereas 94.8% EpCAM positive in the posterior gut cells (FIG. 5). Interestingly, the anterior non-epithelial cells, but not in other populations, highly expressed the RA synthesis gene Aldehyde Dehydrogenase 1 Family Member A2 (ALDHIA2) similar to previous in vivo animal model studies (FIG. 2H). Complementing this, exposing only the posterior gut spheroid, and not the anterior gut spheroid to BMS493 prior to fusion suppressed the protein-level induction of HHEX and PDX1 (FIG. 10). An E9.0 PROX1::GFP reporter mouse embryo, cultured in a whole embryo culture system with BMS493, also displayed significant inhibition of PROX1 expressing cells after 2 days (FIGS. 11A and 11B). Taken together, HBP progenitor self-specification derived from posterior region in boundary organoid model system is regulated by RA signals, and may be supported by co-differentiating anterior non-epithelial, most likely mesenchymal, lineages.

It has been noted that stem cell-derived embryonic endodermal cells are highly plastic and usually generate intestinal tissues 18.25. To examine whether there is an ability to form HBP tissues from progenitors in vivo, transplantations of human PROX1 expressing organoids into immunodeficient mice were performed. One month transplant derived tissues exhibited the small intestinal tissue markers Keratin 20 (CK20), CDX2, and EpCAM, but negligible expression of the other HBP markers (FIG. 21). In addition, the duodenum maker Receptor Accessory Protein 6 (REEP6/DP1L1) and SOX9 expression pattern informed that these human tissues were most similar to duodenum tissue (FIG. 21). These results indicated that despite the presence of HBP progenitors, ectopically transplanted organoids tend to develop intestinal tissues in vivo.

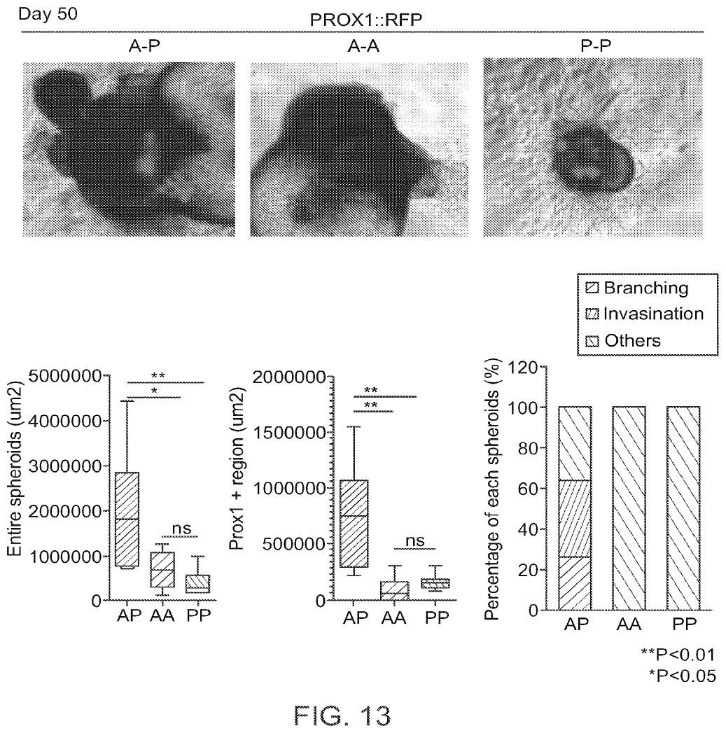

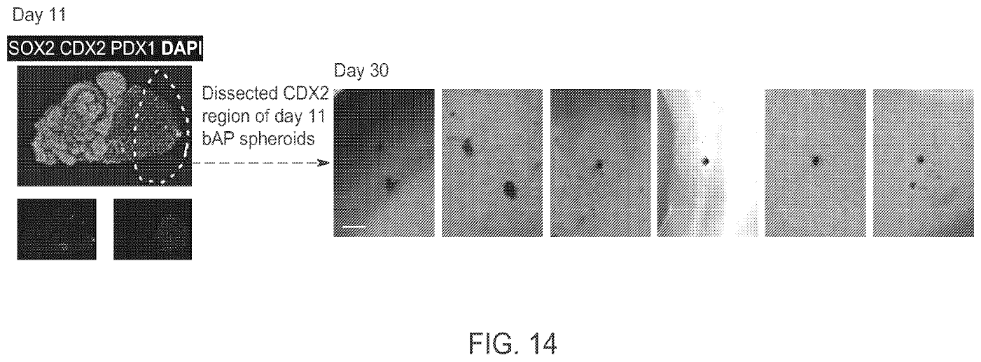

Because the HBP organoids generated predominantly duodenum tissue in vivo, Applicant next excised PROX1 positive regions from the D13 boundary organoids and cultured them in different formats to effectively model HBP organogenesis (FIGS. 3A and B). Strikingly, among the various tested culture conditions (FIG. 12), excised tissues were cultured for two weeks in Matrigel drop on Transwell without specific growth factors; most of the PROX1 tissues developed into spatially organized invaginating epithelium, and formed a branching structure (FIG. 3b). In contrast, only a small number of PROX1 positive epithelium cultured in floating conditions or non-dissected organoids were invaginated (FIG. 3B). Time course imaging showed the dissected PROX1 positive tissue changed from an epithelial morphology into a more convoluted structure during 2 days of culture (FIG. 3C). Around D25, the PROX1 epithelium portion of the organoid started to invaginate. The PROX1 positive region subsequently started to grow outward in multiple directions, forming a branching structure via progressive invagination (FIGS. 3D and D). The branching structures were not observed in A-A and P-P combination (FIG. 13). Furthermore, the posterior gut spheroids alone, which expressed PDX1 initially but dissected from the bAP organoids, were not capable of growing invaginating structures (FIG. 14).

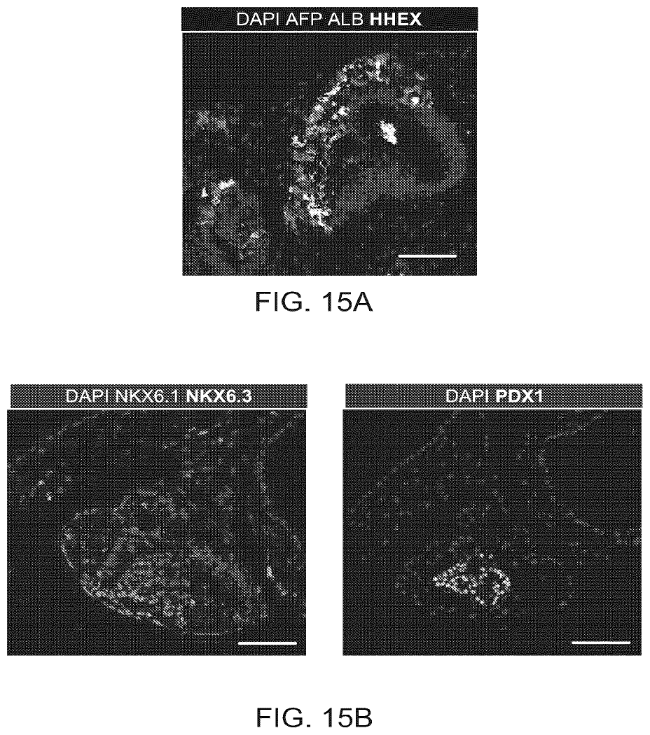

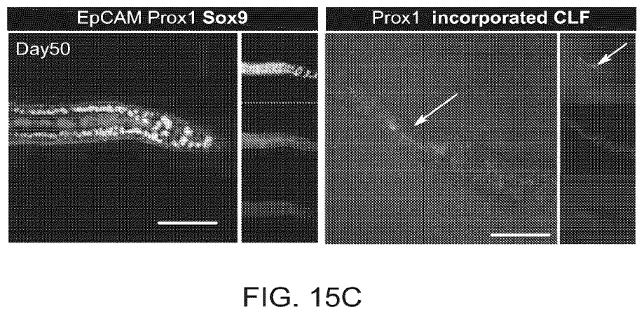

To determine whether longer-term culture can produce more mature tissue, organoids were cultured until D90. The D90 organoids were morphologically similar to mouse E14.5 explanted organ culture (FIG. 3F). H&E staining showed that the long-term cultured organoids morphologically contain liver, pancreas, bile duct and intestine tissues (FIG. 3G). Furthermore, the immunofluorescent staining detected the expression of the pancreas marker PDX1 and NGN3, the liver marker PROX1, and the bile duct markers CK19 and SOX9 in the organoids (FIG. 3H). Alpha-SMA expressing mesenchyme cells wrapped around bile duct SOX17+ cells similar to developing gallbladder tissue²⁶ (FIG. 3h). Immunofluorescent and whole mount staining with liver markers AFP and albumin, pancreatic markers PDX1, NKX6.1 and GATA4, and bile duct markers DBA and SOX9 confirmed that each lineage of tissues segregated from the same boundary organoids after more than 30 days of culture (FIG. 3I-M and FIG. 15A-15B). NKX6.1, HNFIB and GATA4 were differentially expressed on the PDX1 positive region, and pancreatic mesenchymal marker NKX6.3 expression was observed alongside the PDX1 expressing cells, as seen in in vivo developing pancreas (FIG. 3I-M and FIG. 15A-15C). Remarkably, the bile duct and pancreas tissue are directly connected in branching organoids, as evidenced by whole mount co-staining of DBA, SOX9, and PDX1 and by the capacity to incorporate fluorescein-labelled bile acid (CLF)(FIGS. 3K and 3L and FIG. 15C). Moreover, at 90 days, a pancreatic region expressing the exocrine markers amylase and GATA4 was identified in the organoids (FIG. 3N). Given the Cholecystokinin A Receptor (CCKAR) expression in the organoids (FIG. 3P), organoids were exposed to CCK and the pancreatic secretory function was analyzed with an amylase Enzyme-linked immune sorbent assay (ELISA). The ductal structures constricted on the following day, and the CCK treated tissues increased amylase secretion in supernatants compared with untreated controls (FIGS. 3Q and 3R). These results indicate that the boundary organoid strategy not merely generates multiple organ (HBP) tissues but also establishes a functional connection of the pancreas, especially exocrine lineage, and bile duct.

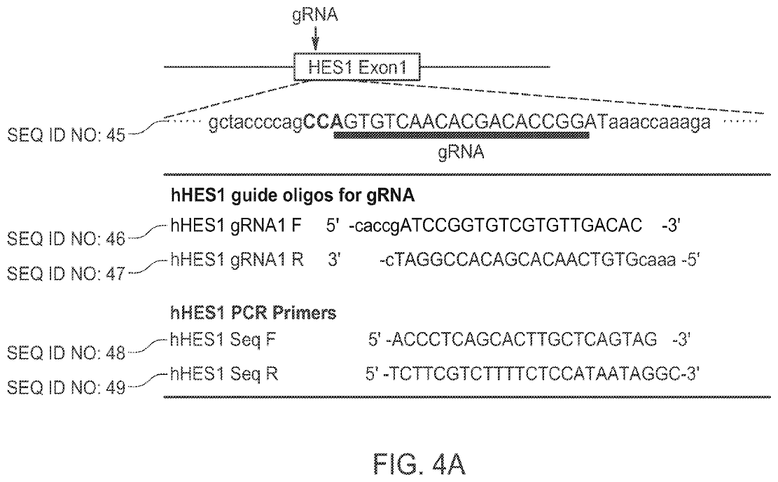

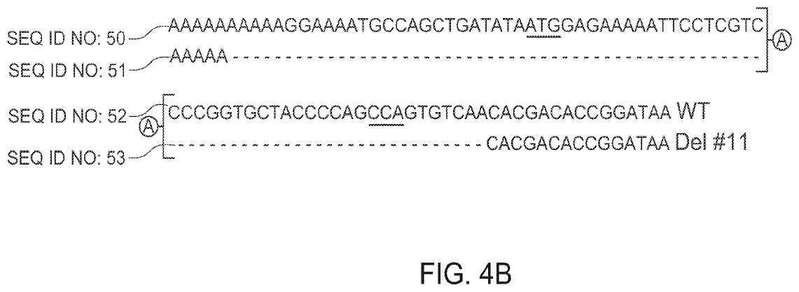

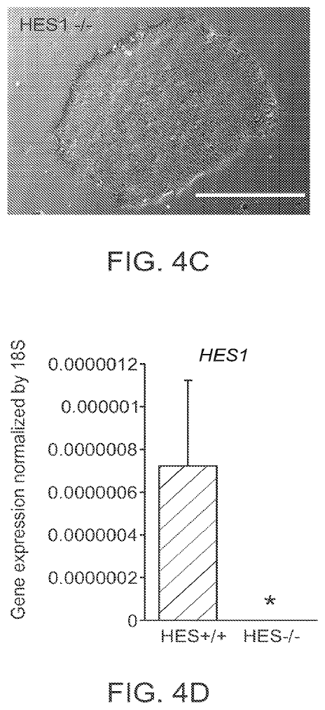

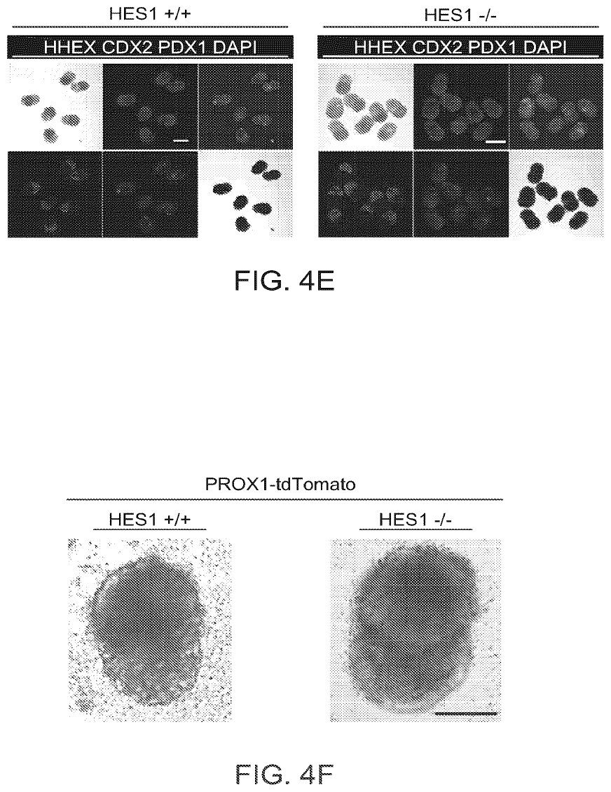

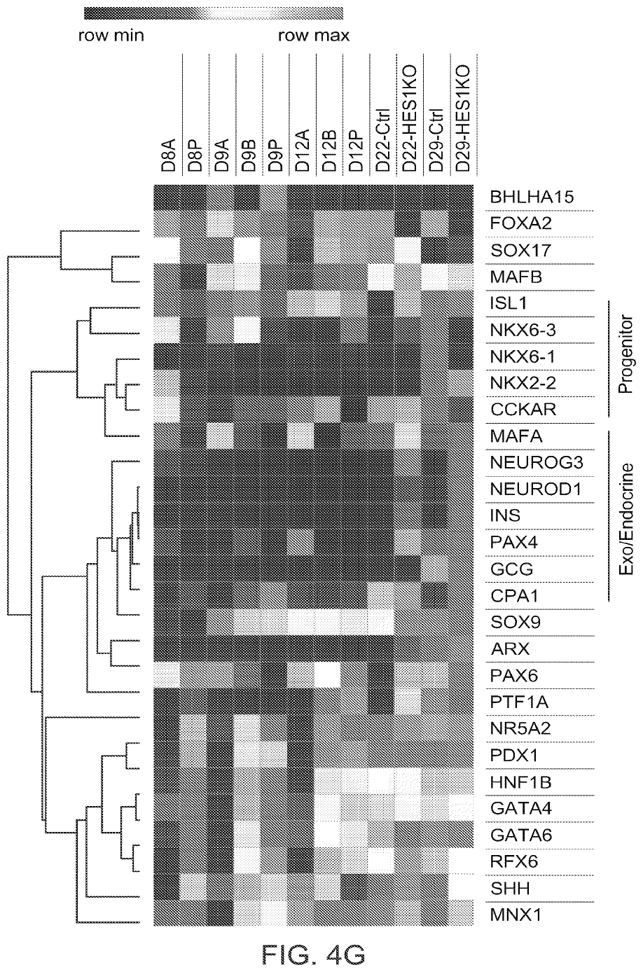

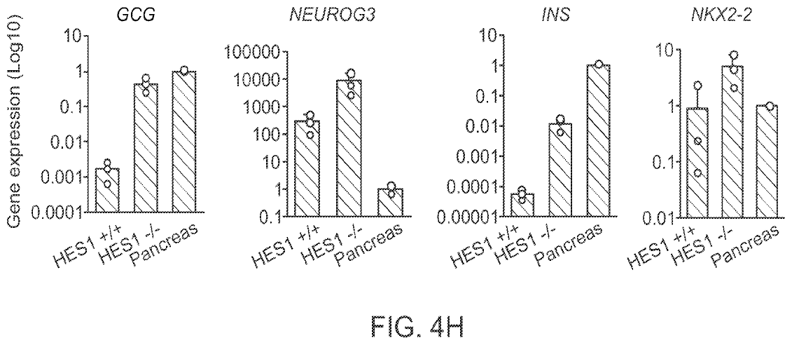

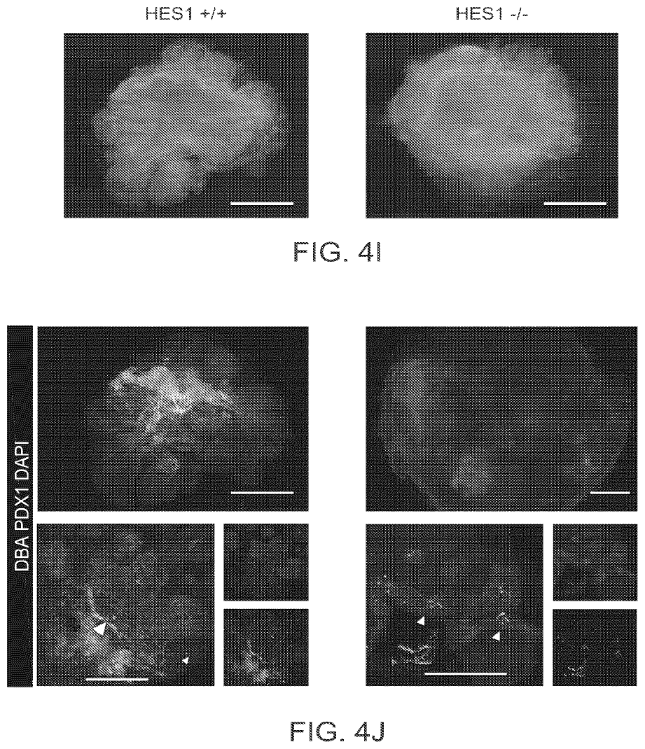

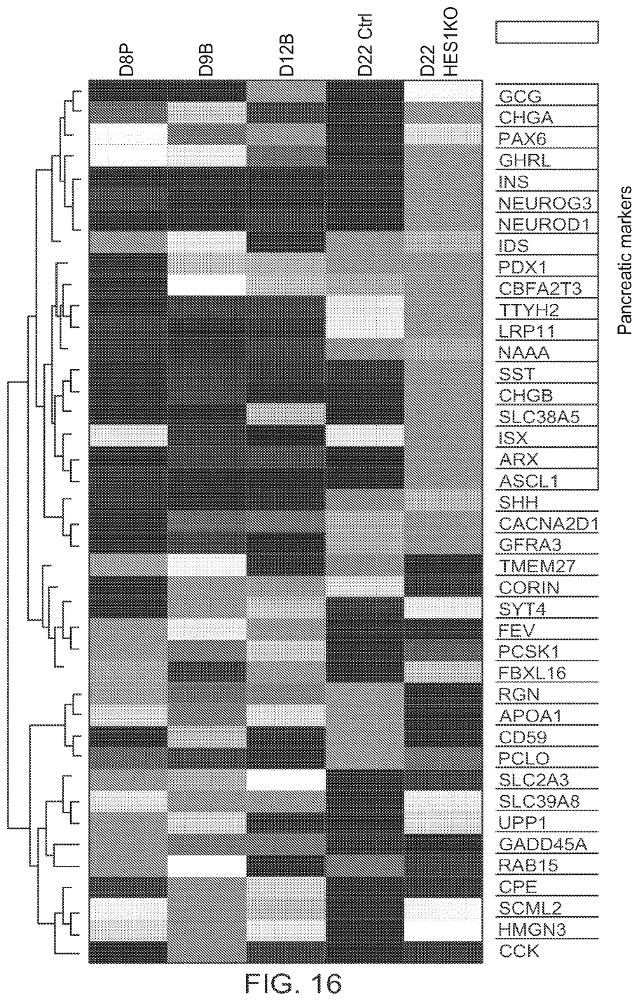

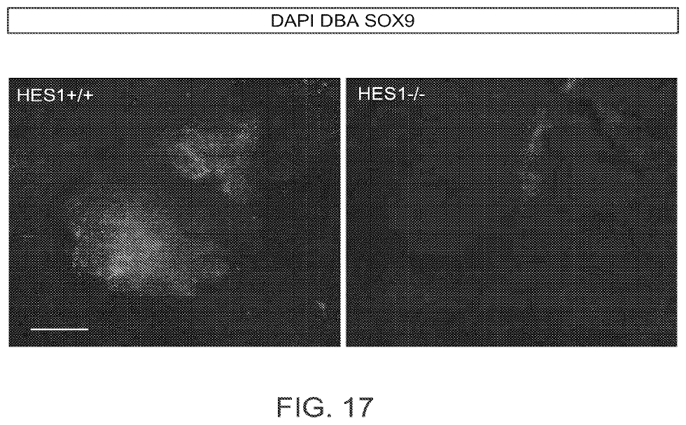

HES1 (Hes family bHLH transcription factor 1) is a transcription factor that regulates the posterior foregut lineage^{15,27}. In Hes1 knock-out rodents, conversion of the biliary system to pancreatic tissue occurs due to failed pancreato-biliary organ segregation^{8,9}. To elucidate whether the HBP organoid recapitulates HES1 mediated developmental process, Applicant established HES1 KO on PROX1 reporter iPSCs by CRISPR/Cas9 system (FIG. 4A-4C), and confirmed the absence of HES1 gene expression in HES1−/− iPSC derived organoids at D20 (FIG. 4D). HES1−/− organoids retained PROX1 reporter activity (FIG. 4E) and HHEX/PDX1 expression at the bAP at D11 (FIG. 4F). RNAseq of HES1−/− and HES1+/+ organoids at Day 22 showed that the significant upregulation of reported pancreatic associated murine genes, including endocrine markers, as HES1 targets^{9,28}, in HES1−/− organoids, compared with HES1+/+organoids (FIG. 4G and FIG. 16). qRT-PCR showed that all the pancreatic gene expression levels of GCG, NEUROG3, INS, and NKX2-2 were upregulated in HES1−/− organoids, compared with HES1+/+ organoids (GCG: 264 fold; NEUROG3: 29.8 fold; INS: 212 fold), however the expression level of GCG, INS were still lower than human pancreases tissue, in both of HES1+/+ and HES1−/− organoids (FIG. 4H). Moreover, consistent with in vivo rodent studies, HES1−/− human organoids produced less DBA and SOX9 positive ductal tissue and more PDX1 positive pancreatic structures compared with HES1+/+ organoids (FIGS. 4I and J and FIG. 17), highlighting the phenotypic relevance to animal model studies.

Multi-organ integration in stem cell culture is a critical unmet challenge. The instant disclosure demonstrates the generation of a human three-dimensional antero-posterior boundary system that leads to structurally and functionally integrated HBP organoids developed at foregut-midgut border.

### Methods

### Maintenance of PSCs

Human PSC lines were maintained as described previously. Undifferentiated hPSCs were maintained on feeder-free conditions in mTeSR1 medium (StemCell technologies, Vancouver, Canada) or Stem Fit medium (Ajinomoto Co, Japan) on plates coated either with Matrigel Growth Factor Reduced (Corning Inc., New York, NY, USA) at 1/30 dilution or iMatrix-511 (Nippi, Japan) at 0.25 ug/cm² in an incubator with 5% CO2/95% air at 37° C.

Differentiation of PSCs into Anterior and Posterior Gut Spheroid

Differentiation of hPSCs into definitive endoderm was induced using previously described methods 18.19 with modifications. In brief, colonies of hiPSCs were isolated in Accutase (Thermo Fisher Scientific Inc., Waltham, MA, USA) and 150,000 cells/mL were plated on Matrigel coated tissue culture plate (VWR Scientific Products, West Chester, PA). Medium was changed to RPMI 1640 medium (Life Technologies, Carlsbad, CA) containing 100 ng/ml Activin A (R&D Systems, Minneapolis, MN) and 50 ng/ml bone morphogenetic protein 4 (BMP4; R&D Systems) at Day 1, 100 ng/ml Activin A and 0.2% fetal calf serum (FCS; Thermo Fisher Scientific Inc.) at Day 2 and 100 ng/mL Activin A and 2% FCS at Day 3. For Day 4-7, cells were cultured in gut growth medium (Advanced DMEM/F12 (Thermo Fisher Scientific Inc.) with 15 mM HEPES, 2 mM L-glutamine, penicillin-streptomycin, B27 (Life Technologies) and N2 (Gibco, Rockville, MD)) supplemented with 200 ng/ml noggin (NOG; R&D Systems), 500 ng/ml fibroblast growth factor 4 (FGF4; R&D Systems) and 2 μM CHIR99021 (Stemgent, Cambridge, MA, USA) for anterior gut cell induction and supplemented with 500 ng/ml FGF 4 and 3 μM CHIR99021 for posterior gut cell induction. Cultures for cell differentiation were maintained at 37° C. in an atmosphere of 5% CO2/95% air and the medium was replaced every day.

### Anterior-Posterior Boundary Spheroid Formation

On Day 7, anterior or posterior gut cells were dissociated to single cells by incubation with TrypLE Express (Life Technologies) at 37° C. Cells were centrifuged at 1000 rpm for 3 minutes and, after removing supernatant, the pellet was re-suspended in gut growth medium containing 10 uM of Y-27632 dihydrochloride (Tocris Bioscience, Bristol, United Kingdom). The anterior or posterior gut cell suspensions were plated on 96 well round bottom ultra-low attachment plate (Corning Inc) at density of 10,000 cells/well and incubated at 37° C. for 24 hours to form spheroid. On Day 8, generated single anterior gut spheroid and posterior gut spheroid were mixed on 96 well round bottom ultra-low attachment plate in gut growth medium for 24 hours to form fused boundary spheroids (A-P spheroids).

### Hepato-Biliary-Pancreatic (HBP) Organoid Culture and Transplantation

On Day 9, A-P spheroids were embedded in Matrigel drop and were cultured in gut growth medium to generate multi-organ HBP organoids. For longer-term culture, HBP organoids were dissected and/or transferred to transwell for air-liquid interface culture at Day 13. Cultures for spheroid were maintained at 37° C. in an atmosphere of 5% CO2/95% air and the gut growth medium were replaced every 4 days. Single HBP organoid at Day 13 was transplanted into the subcapsule of the kidney in male immune deficient NSG (NOD.Cg-Prkdc_{scid}Il2rg^{tmlWjl}/SzJ) mice, aged 12 weeks old. All experiments were performed under the approval of the Institutional Animal Care and Use Committee of CCHMC (protocols IACUC2018-0096).

### H&E Staining and Immunohistochemistry

Spheroid and organoid were collected from Matrigel, fixed in 4% paraformaldehyde (PFA) and embedded in paraffin. Sections were subjected to H&E and immunohistochemical staining. The primary antibodies were listed in Table 1. Immunohistochemical staining was performed by using ultra View Universal DAB Detection Kit (Roche Diagnostics, Basel, Switzerland). The specimens were observed under a microscope.

For whole mount immunohistochemical staining, spheroid and organoid were collected from Matrigel and removed remaining Matrigel by treating with Cell recovery solution at 4° C. for 30 min. The tissues were washed by PBS and were fixed in 4% PFA at 4° C. for overnight. The fixed samples were treated by 4% PFA with 0.5% Triton X100 at room temperature for 15 min and permeabilized with 0.1% Tween 20 (Sigma) at room temperature for 15 min. The samples were treated with blocking solution (1% BSA, 0.3% Triton X100) at room temperature for 1 hour and were incubated overnight at 4° C. with the primary antibodies diluted in blocking solution. After washing, fluorescent dye-conjugated secondary antibodies were applied to the samples at room temperature for 2 hours. The primary and secondary antibodies are listed in Table 1. After the secondary antibody reaction, the samples were washed three times. Nuclei were stained with DAPI mounting solution.

The stained section and whole mount samples were observed under a Nikon A1Rsi inverted confocal microscope.

### RNA Isolation, RT-qPCR

RNA was isolated using the RNeasy mini kit (Qiagen, Hilden, Germany). Reverse transcription was carried out using the SuperScript IV First-Strand Synthesis System for RT-PCR (Invitrogen, CA, USA) according to manufacturer's protocol. qPCR was carried out using TaqMan gene expression master mix (Applied Biosystems) on a QuantStudio 3 Real-Time PCR System (Thermo). All primers and probe information for each target gene was obtained from the Universal ProbeLibrary Assay Design Center (https://qpcr.probefinder.com/organism.jsp) and listed in Table 2.

### RNA Sequencing

Sample preparation for RNA sequencing was performed using SMART-seq v4 Ultra Low Input RNA Kit for Sequencing (Clontech Laboratories) according manufacture's user manual.

Briefly, First-strand cDNA synthesis was primed by the 3′ SMART-seq CDS Primer II A and uses the SMART-Seq v4 Oligonucleotide for template switching at the 5′ end of the transcript. PCR Primer II A amplified cDNA from the SMART sequences introduced by 3′ SMART-Seq CDS Primer II A and the SMART-Seq v4 Oligonucleotide by PCR. PCR-amplified cDNA was purified by immobilization on AMPure XP beads. The beads were then washed with 80% ethanol and cDNA was eluted with Elution Buffer. Amplified cDNA was validated using the Agilent 2100 Bioanalyzer and Agilent's High Sensitivity DNA Kit (Agilent) according Kit User Manual. The full-length cDNA output of the SMART-Seq v4 Ultra Low Input RNA Kit for Sequencing was processed with the Nextera XT DNA Library Preparation Kit (Illumina).

The RNA profiles were compiled with Kallisto software and expressed as transcripts per million (TPM). The data sets filtered by the threshold requiring greater than O in at least 1 sample were first subjected to gene functional classification based on gene sets related to FGF, BMP, Hedgehog, NOTCH and RA signaling pathway. The lists of gene sets were acquired from Molecular Signatures Database (ver 6.2). Unbiased cluster analysis for filtered data sets performed by using Cluster 3.0 software. Detail of gene sets included in each cluster were listed in Table 3.

### Flow Cytometry.

For flow cytometry, Anterior gut cells were differentiated from GFP-Labeled iPSCs and posterior gut cells were differentiated from mCherry-Labeled iPSCs. At day 13, A-P spheroid were dissociated to single cells by the treatment of TrypLE Express for 10 min at 37° C. After PBS wash, the single cells were incubated with BV421-conjugated EpCAM antibody (BioLegend) at room temperature for 30 min. After PBS wash, cell sorting was performed by BD FACS AriaII (BD Biosciences). Analysis was performed by BD FACS DIVA software and FlowJo (FlowJo, LLC).

### CRISPR Editing

The plasmid encoding Cas9-2A-GFP was acquired from addgene (#44719, doi: 10.1016/j.stem.2013.03.006). Guide RNA targeting the N-terminus of PROX1 or HES1 was synthesized by Integrated DNA Technologies, cloned into the pGL3-U6-sgRNA-PGK-puromycin vector (addgene #51133, doi: 10.1038/nmeth.2857) and sequenced using the RV3 universal primer. To construct the HDR template, homology arms flanking the PROX1 start codon were independently amplified from genomic DNA and then fused to tdTomato via overlap extension PCR using the high-fidelity taq polymerase iProof (Bio-Rad). The resulting PCR product was then cloned into the pCR-Blunt II-TOPO cloning vector (Invitrogen) and confirmed by Sanger sequencing.

Human iPSCs were transfected with 2 ug of each plasmid using the Lipofectamine 3000 following the manufacturer's instructions. Twenty-four hours after transfection, cells were sorted by GFP expression to select for positively transfected cells. Clonal cells were expanded for 2 weeks and screened for inserted PROX1-tdTomato or depleted HES1 exon1 sequence and karyotyped.

### Amylase ELISA.

To measure Amylase secretion level of organoids, 200 μL of culture supernatant was collected from organoid embedded in Matrigel. The culture supernatants were collected at 48 hrs time points after the culture and stored at −80° C. until use. The supernatant was centrifuged at 1,500 rpm for 3 min and to pellet debris, and the resulting supernatant was assayed with Human Amylase ELISA Kit according to the manufacturer's instructions.

### Statistics and Reproducibility.

Statistical analysis was performed using unpaired two-tailed Student's t-test, Dunn-Holland-Wolfe test, or Welch's t-test. Results were shown mean±s.d.; P values <0.05 were considered statistically significant. N-value refers to biologically independent replicates, unless noted otherwise. For comparisons between unpaired 2 groups, non-parametric Brunner-Munzel test was performed when 2 samples were independent, and the variances of the samples were unequal. For comparisons between more than 2 samples, non-parametric Kruskal-Wallis and post hoc Dunn-Holland-Wolf test were performed.

### Mouse Whole Embryo Culture

The culturing of the multi-organ three-dimensional organoid may be carried out using a whole embryo culture system (Ikemoto Scientific Technology, Tokyo, Japan) with improved growth. The multi-organ three-dimensional organoid may be transferred into a sterile glass roller bottle containing culture media and the flask closed by silicon plug. The number of transferred organoids depends on how long they will be cultured. The culture bottles may be attached to a rotator drum and rotated at 20 rpm and 37° C. in the dark while being continuously supplied with a gas mixture (mainly 20% O₂, 5% CO₂ and 75% N2, but the O₂ concentration can be modified with balanced with N2). The organoids can be cultured for a few weeks (with the longest trial being 10 weeks). The culture media is changed every 5-7 days and the viability and growth can be monitored based on the organoid length and media components such as Alb, Amy, C-peptide levels.

The Rotator-type Bottle Culture System (Ikemoto Scientific Technology Co., Ltd) was used for whole embryo culture. E9.0 Prox1-GFP mouse embryo was dissected and transfer to culture bottle with Advanced DMEM/F12 supplemented B27 and N2 supplements. The temperature inside the whole embryo culture system was kept at 37.0° C.

TABLE 2
Antibodies used in this study

| Target | Company | Catalog |
|---|---|---|
| SOX2 | Seven Hills | WRAB-1236 |
| CDX2 | Biogenex | MU392A-UC |
| PDX1 | Abcam | ab47308 |
| HHEX | R&D | MAB83771 |
| E-Cadherin | R&D | AF648 |
| E-Cadherin | BD Pharmingen | 610182 |
| RFP | Rockland | RL600-401-379S |
| REEP6 | Proteintech | 12088-1-AP |
| REEP6 | Novus | NBP2-37919 |
| SOX9 | R&D | AF3075 |
| Insulin | eBioscience | 14-9769 |
| Amylase | Sigma-Aldrich | HPA045394 |
| GATA4 | Santa cruz | sc-25310 |
| GATA4 | Abcam | ab134057 |
| Cytokeratin 19 | eBioscience | 14-9898 |
| Neurogenin 3 | R&D | MAB3444 |
| PROX1 | Abcam | ab38692 |
| PROX1 | Millipore | AB5475 |
| Alpha smooth muscle Actin | Abcam | ab5694 |
| SOX17 | R&D | AF1924 |
| SOX17 | R&D | MAB1924 |
| Alpha-Fetoprotein | eBioscience | 14-9499 |
| EpCAM | R&D | AF960 |
| NKX6.1 | Novus | NBP1-49672SS |
| Albumin | Sigma-Aldrich | A6684 |
| CD31 | eBioscience | 14-0318 |
| CD34 | Abcam | ab198395 |
| HNF1B | Santa cruz | sc-7411 |
| CCKAR | Novus | AF2680 |
| NKX6-3 | Sigma-Aldrich | HPA042790 |

TABLE 3
Primers for qPCR used in this study

| Gene | Probe | 5′ primer | 3′ primer |
|---|---|---|---|
| SOX2 | 65 | gggggaatggaccttgtatag | gcaaagctcctaccgtacca |
| | | (SEQ ID NO: 1) | (SEQ ID NO: 2) |
| CDX2 | 34 | atcaccatccggaggaaag | tgcggttctgaaaccagatt |
| | | (SEQ ID NO: 3) | (SEQ ID NO: 4) |
| PDX1 | 78 | aagctcacgcgtggaaag | gccgtgagatgtacttgttgaa |
| | | (SEQ ID NO: 5) | (SEQ ID NO: 6) |
| HHEX | 61 | cggacggtgaacgactaca | agaaggggctccagagtagag |
| | | (SEQ ID NO: 7) | (SEQ ID NO: 8) |
| ALDH1A2 | 63 | Ccacagtgattccaacgtc | tcctgaacagggccaaag |
| | | (SEQ ID NO: 9) | (SEQ ID NO: 10) |
| CYP26A1 | 28 | Cgagcactcgtgggagag | ccaaagaggagttcggttga |
| | | (SEQ ID NO: 11) | (SEQ ID NO: 12) |
| RARA | 67 | Gccatctgcctcatctgc | tccgcacgtagacctttagc |
| | | (SEQ ID NO: 13) | (SEQ ID NO: 14) |
| RARB | 53 | Ccgaaaagctcaccagga | cgatggtcagcactggaat |
| | | (SEQ ID NO: 15) | (SEQ ID NO: 16) |
| RARG | 24 | Cagccctacatgttcccaag | ggcctggaatctccatcttc |
| | | (SEQ ID NO: 17) | (SEQ ID NO: 18) |
| HES1 | 20 | tgccagctgatataatggagaa | ctccataataggctttgatgacttt |
| | | (SEQ ID NO: 19) | (SEQ ID NO: 20) |
| INS | 27 | aggcttcttctacacacccaag | Cacaatgccacgcttctg |
| | | (SEQ ID NO: 21) | (SEQ ID NO: 22) |
| GCG | 82 | gtacaaggcagctggcaac | Tgggaagctgagaatgatctg |
| | | (SEQ ID NO: 23) | (SEQ ID NO: 24) |
| NKX2-2 | 71 | cgagggccttcagtactcc | ggggacttggagcttgagt |
| | | (SEQ ID NO: 25) | (SEQ ID NO: 26) |
| CPA1 | 37 | cagcatcatcaaggcaatttat | ggagctcgaaggtgaagga |
| | | (SEQ ID NO: 27) | (SEQ ID NO: 28) |
| CEL | 73 | caccttcaactaccgtgtcg | gatcccgaaggccatagttac |
| | | (SEQ ID NO: 29) | (SEQ ID NO: 30) |
| CFTR | 52 | gaagtagtgatggagaatgtaacagc | gctttctcaaataattccccaaa |
| | | (SEQ ID NO: 31) | (SEQ ID NO: 32) |
| CAPN6 | 4 | aggctgctataacaaccgtga | catcctcaggcacagtgaag |
| | | (SEQ ID NO: 33) | (SEQ ID NO: 34) |
| ALB | 7 | gtgaggttgctcatcggttt | gagcaaaggcaatcaacacc |
| | | (SEQ ID NO: 35) | (SEQ ID NO: 36) |
| CYP3A7 | 2 | caaacttggccgtggaaa | agtccatgtgtacgggttcc |
| | | (SEQ ID NO: 37) | (SEQ ID NO: 38) |
| HNF4A | 68 | gagatccatggtgttcaagga | gtgccgagggacaatgtagt |
| | | (SEQ ID NO: 39) | (SEQ ID NO: 39) |
| AFP | 61 | tgtactgcagagataagtttagctgac | tccttgtaagtggcttcttgaac |
| | | (SEQ ID NO: 41) | (SEQ ID NO: 42) |
| APOA2 | 68 | gagaaggtcaagagcccaga | ccttcttgatcaggggtgtc |
| | | (SEQ ID NO: 43) | (SEQ ID NO: 44) |

### REFERENCES

1 Smith, D. M., Nielsen, C., Tabin, C. J. & Roberts, D. J. Roles of BMP signaling and Nkx2.5 in patterning at the chick midgut-foregut boundary. Development 127, 3671-3681 (2000).
2 Hentsch, B. et al. Hlx homeo box gene is essential for an inductive tissue interaction that drives expansion of embryonic liver and gut. Genes Dev 10, 70-79 (1996).
3 San Roman, A. K. & Shivdasani, R. A. Boundaries, junctions and transitions in the gastrointestinal tract. Exp Cell Res 317, 2711-2718, doi: 10.1016/j.yexcr.2011.07.011 (2011).
4 Nielsen, C., Murtaugh, L. C., Chyung, J. C., Lassar, A. & Roberts, D. J. Gizzard formation and the role of Bapx1. Dev Biol 231, 164-174, doi: 10.1006/dbio.2000.0151 (2001).
5 Bort, R., Martinez-Barbera, J. P., Beddington, R. S. & Zaret, K. S. Hex homeobox gene-dependent tissue positioning is required for organogenesis of the ventral pancreas. Development 131, 797-806, doi: 10.1242/dev.00965 (2004).
6 Shih, H. P. et al. A Gene Regulatory Network Cooperatively Controlled by Pdx1 and Sox9 Governs Lineage Allocation of Foregut Progenitor Cells. Cell Rep 13, 326-336, doi: 10.1016/j.celrep.2015.08.082 (2015).
7 Tepass, U. & Hartenstein, V. Epithelium formation in the Drosophila midgut depends on the interaction of endoderm and mesoderm. Development 120, 579-590 (1994).
8 Fukuda, A. et al. Ectopic pancreas formation in Hes1-knockout mice reveals plasticity of endodermal progenitors of the gut, bile duct, and pancreas. J Clin Invest 116, 1484-1493, doi: 10.1172/JCI27704 (2006).
9 Sumazaki, R. et al. Conversion of biliary system to pancreatic tissue in Hes1-deficient mice. Nat Genet 36, 83-87, doi: 10.1038/ng1273 (2004).
10 Udager, A., Prakash, A. & Gumucio, D. L. Dividing the tubular gut: generation of organ boundaries at the pylorus. Prog Mol Biol Transl Sci 96, 35-62, doi: 10.1016/B978-O-12-381280-3.00002-6 (2010).
11 Zhang, Z., Rankin, S. A. & Zorn, A. M. Syndecan4 coordinates Wnt/JNK and BMP signaling to regulate foregut progenitor development. Dev Biol 416, 187-199, doi: 10.1016/j.ydbio.2016.05.025 (2016).
12 McLin, V. A., Rankin, S. A. & Zorn, A. M. Repression of Wnt/beta-catenin signaling in the anterior endoderm is essential for liver and pancreas development. Development 134, 2207-2217, doi: 10.1242/dev.001230 (2007).
13 Nissim, S. et al. Iterative use of nuclear receptor Nr5a2 regulates multiple stages of liver and pancreas development. Dev Biol 418, 108-123, doi: 10.1016/j.ydbio.2016.07.019 (2016).
14 Palaria, A., Angelo, J. R., Guertin, T. M., Mager, J. & Tremblay, K. D. Patterning of the hepato-pancreatobiliary boundary by BMP reveals heterogeneity within the murine liver bud. Hepatology 68, 274-288, doi: 10.1002/hep.29769 (2018).
15 Spence, J. R. et al. Sox 17 regulates organ lineage segregation of ventral foregut progenitor cells. Dev Cell 17, 62-74, doi: 10.1016/j.devcel.2009.05.012 (2009).
16 Ameri, J. et al. FGF2 specifies hESC-derived definitive endoderm into foregut/midgut cell lineages in a concentration-dependent manner. Stem Cells 28, 45-56, doi: 10.1002/stem.249 (2010).
17 Zhang, R. R. et al. Human iPSC-Derived Posterior Gut Progenitors Are Expandable and Capable of Forming Gut and Liver Organoids. Stem Cell Reports 10, 780-793, doi: 10.1016/j.stemcr.2018.01.006 (2018).
18 Spence, J. R. et al. Directed differentiation of human pluripotent stem cells into intestinal tissue in vitro. Nature 470, 105-109, doi: 10.1038/nature09691 (2011).
19 McCracken, K. W. et al. Modelling human development and disease in pluripotent stem-cell-derived gastric organoids. Nature 516, 400-404, doi: 10.1038/nature13863 (2014).
20 Wang, Z., Dollé, P., Cardoso, W. V. & Niederreither, K. Retinoic acid regulates morphogenesis and patterning of posterior foregut derivatives. Dev Biol 297, 433-445, doi: 10.1016/j.ydbio.2006.05.019 (2006).
21 Rankin, S. A. et al. Timing is everything: Reiterative Wnt, BMP and RA signaling regulate developmental competence during endoderm organogenesis. Dev Biol 434, 121-132, doi: 10.1016/j.ydbio.2017.11.018 (2018).
22 Cunningham, T. J. & Duester, G. Mechanisms of retinoic acid signalling and its roles in organ and limb development. Nat Rev Mol Cell Biol 16, 110-123, doi: 10.1038/nrm3932 (2015).
23 Rankin, S. A. et al. A Retinoic Acid-Hedgehog Cascade Coordinates Mesoderm-Inducing Signals and Endoderm Competence during Lung Specification. Cell Rep 16, 66-78, doi: 10.1016/j.celrep.2016.05.060 (2016).
24 Bayha, E., Jørgensen, M. C., Serup, P. & Grapin-Botton, A. Retinoic acid signaling organizes endodermal organ specification along the entire antero-posterior axis. PLOS One 4, e5845, doi: 10.1371/journal.pone.0005845 (2009).
25 Watson, C. L. et al. An in vivo model of human small intestine using pluripotent stem cells. Nat Med 20, 1310-1314, doi: 10.1038/nm.3737 (2014).
26 Higashiyama, H. et al. Embryonic cholecystitis and defective gallbladder contraction in the. Development 144, 1906-1917, doi: 10.1242/dev.147512 (2017).
27 Thamm, K. & Seaver, E. C. Notch signaling during larval and juvenile development in the polychaete annelid Capitella sp. I. Dev Biol 320, 304-318, doi: 10.1016/j.ydbio.2008.04.015 (2008).
28 Jørgensen, M. C. et al. Neurog3-dependent pancreas dysgenesis causes ectopic pancreas in. Development 145, doi: 10.1242/dev.163568 (2018).
29 Camp, J. G. et al. Multilineage communication regulates human liver bud development from pluripotency. Nature 546, 533-538, doi: 10.1038/nature22796 (2017).
30 Takebe, T. et al. Vascularized and functional human liver from an iPSC-derived organ bud transplant. Nature 499, 481-484, doi: 10.1038/nature12271 (2013).
31 Davenport, C., Diekmann, U., Budde, I., Detering, N. & Naujok, O. Anterior-Posterior Patterning of Definitive Endoderm Generated from Human Embryonic Stem Cells Depends on the Differential Signaling of Retinoic Acid, Wnt-, and BMP-Signaling. Stem Cells 34, 2635-2647, doi: 10.1002/stem.2428 (2016).
32 Green, M. D. et al. Generation of anterior foregut endoderm from human embryonic and induced pluripotent stem cells. Nat Biotechnol 29, 267-272, doi: 10.1038/nbt. 1788 (2011).

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

What is claimed is:

1. An in vitro hepato-biliary-pancreatic organoid (“HBPO”), wherein said HBPO has two or more functions selected from hepatic tissue function, biliary tissue function, exocrine pancreatic function, and endocrine pancreatic tissue function.

2. The HBPO of claim 1, wherein said HBPO comprises an anterior region, a posterior region and a boundary region, wherein said boundary region expresses Pancreatic and Duodenal Homeobox 1 (PDX1) and Hematopoietically-Expressed Homeobox Protein (HHEX).

3. The HBPO of claim 1, wherein said HBPO comprises bile duct tissue and pancreatic tissue.

4. The HBPO of claim 3, wherein said bile duct tissue and said pancreatic tissue are connected.

5. The HBPO of claim 1, wherein said HBPO comprises liver cells, pancreas cells, bile duct cells, and intestinal cells.

6. The HBPO of claim 1, wherein said HBPO comprises endothelial cells.

7. The HBPO of claim 1, wherein said HBPO comprises mesenchymal cells.

8. The HBPO of claim 1, wherein said HBPO comprises endoderm and mesoderm.

9. The HBPO of claim 1, wherein said HBPO has a branched structure.

10. The HBPO of claim 1, wherein said HBPO expresses a functional exocrine marker, preferably amylase, and an endocrine marker, preferably insulin.

11. The HBPO of claim 1, wherein said HBPO secretes amylase in response to a hormone,

12. The HBPO of claim 11, wherein said hormone is cholecystokinin (CCK).

13. The HBPO of claim 1, wherein said HBPO is substantially free of one or more of submucosal glands, transition zones, vasculature, immune cells, or submucosal layers.

14. The HBPO of claim 1, wherein said HBPO is obtained by expansion of one or more precursor cells.
